(19) United States
(12) Patent Application Publication
(10) Pub. No.: US 2026/0102584 A1
(43) Pub. Date: Apr. 16, 2026

(54) SYSTEMS AND METHODS FOR PROVIDING SOUND AND VIBRATIONAL THERAPY

(71) Applicant: MODERN HYGIENE, INC., Middletown, DE (US)

(72) Inventor: Ray Kelly, Brooklyn, NY (US)

(21) Appl. No.: 19/116,135

(22) PCT Filed: Sep. 29, 2023
(86) PCT No.: PCT/US2023/075595

§ 371 Date: Mar. 27, 2025

Related U.S. Application Data
(60) Provisional application No. 63/377,842, filed on Sep. 30, 2022.

(51) Int. Cl. A61M 21/02; A61M 21/00

(52) U.S. Cl. CPC A61M 21/02; A61M 2021/0016; A61M 2021/0022; A61M 2021/0027; A61M 2205/0216; A61M 2205/502; A61M 2209/084; A61M 2210/0662

(57) ABSTRACT

Systems and methods for providing vibrational and sound therapy routines to users, optionally determined based on a user's physiological and/or emotional characteristics through the application of one or more artificial intelligence or machine learning algorithms which can be based on collected data. The system may comprise: a bed portion configured to receive and support a user; a plurality of transducers positioned below the bed portion, wherein the plurality of transducers are configured to produce vibrations; and a plurality of speakers.

### INCORPORATION BY REFERENCE TO ANY PRIORITY APPLICATIONS

The present application claims priority benefit to U.S. Provisional Application No. 63/377,842, filed Sep. 30, 2022, entitled “PROVIDING SOUND AND VIBRATIONAL THERAPY”, which is hereby incorporated herein by reference in its entirety. Any and all applications for which a foreign or domestic priority claim is identified in the Application Data Sheet as filed with the present application are incorporated by reference under 37 CFR 1.57 and made a part of this specification.

### LIMITED COPYRIGHT AND IP AUTHORIZATION

A portion of the disclosure of this patent document includes material which is subject to copyright protection and/or other IP protection. The copyright/IP owner has no objection to the facsimile reproduction by anyone of the patent document or the patent disclosure as it appears in the Patent and Trademark Office patent file or records, but otherwise reserves all copyrights and/or IP whatsoever.

### TECHNICAL FIELD

This application is directed to devices, systems, and methods for sound, vibrational, and/or visual therapy for users.

### SUMMARY

The systems, methods, and devices described herein each have several aspects, no single one of which is solely responsible for its desirable attributes. Without limiting the scope of this disclosure, several non-limiting features will now be described briefly.

The systems, methods, and devices described herein are configured to provide users or consumers the ability to receive vibrational and sound therapy at home with therapy routines that are focused on improving the emotional state, brain synchronization, and mood.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings and the associated descriptions are provided to illustrate embodiments of the present disclosure and do not limit the scope of the claims. Aspects and many of the attendant advantages of this disclosure will become more readily appreciated as the same become better understood by reference to the following detailed description, when taken in conjunction with the accompanying drawings.

FIG. 1 is a front, top, and right-side perspective view of a therapy bed, according to various embodiments of the present disclosure.

FIG. 2 is a front view of the therapy bed of FIG. 1.

FIG. 3 is a back view of the therapy bed of FIG. 1.

FIG. 4 is a left-side view of the therapy bed of FIG. 1.

FIG. 5 is a ride-side view of the therapy bed of FIG. 1.

FIG. 6 is a top view of the therapy bed of FIG. 1.

FIG. 7 is a bottom view of the therapy bed of FIG. 1.

FIG. 8 is a section view of the therapy bed of FIG. 1 along the line 1A-1A in FIG. 2.

FIG. 9 is a section view of the therapy bed of FIG. 1 along the line 1B-1B in FIG. 2.

FIG. 10 is a section view of the therapy bed of FIG. 1 along the line 1C-1C in FIG. 2.

FIG. 11 is an exploded front, top, and right-side perspective view of FIG. 1 that shows an example of the therapy bed and its internal layering, according to various embodiments of the present disclosure.

FIG. 12A is an overall system diagram illustrating an embodiment of a therapy environment, according to various embodiments of the present disclosure.

FIG. 12B illustrates an embodiment of a therapy system and system subcomponents, according to various embodiments of the present disclosure.

FIG. 13A illustrates an example brain before and after brainwave synchronization, according to various embodiments of the present disclosure.

FIG. 13B illustrates example binaural beats that may be delivered to a user, according to various embodiments of the present disclosure.

FIGS. 14A-14J illustrate example interactive graphical user interfaces related to emotional intelligence-based vibrational and sound therapy, according to various embodiments of the present disclosure.

FIG. 15 illustrates a flow diagram of an embodiment of a method of determining music for a therapy routine based on an emotional state of a user.

FIG. 16 illustrates a block diagram depicting an embodiment of a computer hardware system configured to run software for implementing one or more embodiments disclosed herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### I. Overview

Embodiments of the disclosure will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments of the disclosure. Furthermore, embodiments of the disclosure may include several novel features, no single one of which is solely responsible for its desirable attributes or which is essential to practicing the embodiments of the disclosure herein described.

### II. Terms

In order to facilitate an understanding of the systems and methods discussed herein, a number of terms are defined below. The terms defined below, as well as other terms used herein, should be construed broadly to include the provided definitions, the ordinary and customary meaning of the terms, and/or any other implied meaning for the respective terms. Thus, the definitions below do not limit the meaning of these terms, but only provide example definitions.

User Input (also referred to as “Input”): Any interaction, data, indication, etc., received by a system/device from a user, a representative of a user, an entity associated with a user, and/or any other entity. Inputs may include any interactions that are intended to be received and/or stored by the system/device; to cause the system/device to access and/or store data items; to cause the system to analyze, integrate, and/or otherwise use data items; to cause the system to update data that is displayed; to cause the system to update a way that data is displayed; to transmit or access data; and/or the like. Non-limiting examples of user inputs include keyboard inputs, mouse inputs, digital pen inputs, voice inputs, finger touch inputs (e.g., via touch sensitive display), gesture inputs (e.g., hand movements, finger movements, arm movements, movements of any other appendage, and/or body movements), and/or the like. Additionally, user inputs to the system may include inputs via tools and/or other objects manipulated by the user. For example, the user may move an object, such as a tool, stylus, or wand, to provide inputs. Further, user inputs may include motion, position, rotation, angle, alignment, orientation, configuration (e.g., fist, hand flat, one finger extended, etc.), and/or the like. For example, user inputs may comprise a position, orientation, facial expression, and/or motion of a hand or other appendage, article, a body, a 3D mouse, and/or the like.

Data Store: Any computer readable storage medium and/or device (or collection of data storage mediums and/or devices). Examples of data stores include, but are not limited to, optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. Another example of a data store is a hosted storage environment that includes a collection of physical data storage devices that may be remotely accessible and may be rapidly provisioned as needed (commonly referred to as “cloud”storage).

Database: Any data structure (and/or combinations of multiple data structures) for storing and/or organizing data, including, but not limited to, relational databases (e.g., Oracle databases, PostgreSQL databases, etc.), non-relational databases (e.g., NoSQL databases, etc.), in-memory databases, spreadsheets, comma separated values (CSV) files, eXtendible markup language (XML) files, TeXT (TXT) files, flat files, spreadsheet files, and/or any other widely used or proprietary format for data storage. Databases are typically stored in one or more data stores. Accordingly, each database referred to herein (e.g., in the description herein and/or the figures of the present application) is to be understood as being stored in one or more data stores. Additionally, although the present disclosure may show or describe data as being stored in combined or separate databases, in various embodiments such data may be combined and/or separated in any appropriate way into one or more databases, one or more tables of one or more databases, etc. As used herein, a data source may refer to a table in a relational database, for example.

### III. Hardware

The inventions disclosed herein are described below in the context of a home therapy system for supporting vibrational and sound therapy because they have particular utility in this context. However, the inventions disclosed herein are applicable to other contexts as well.

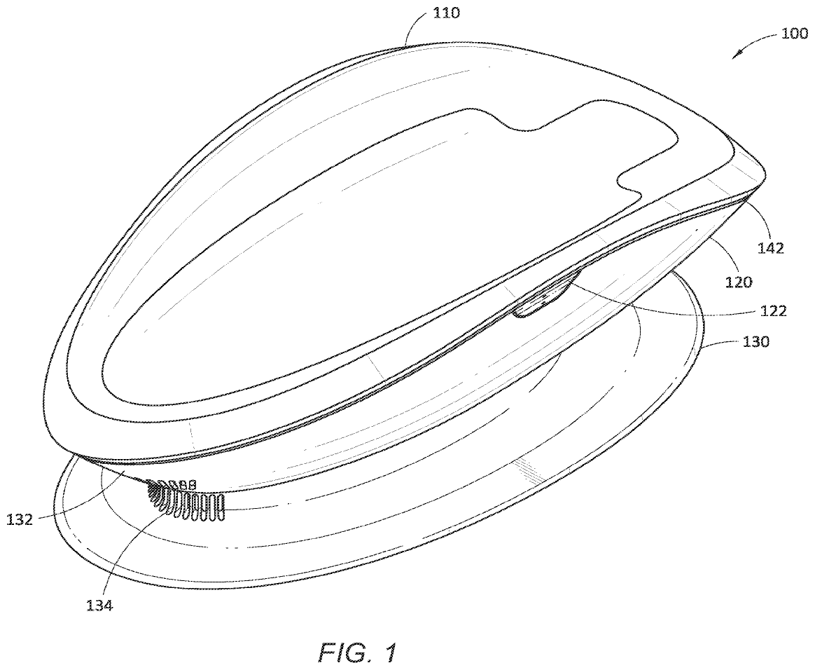

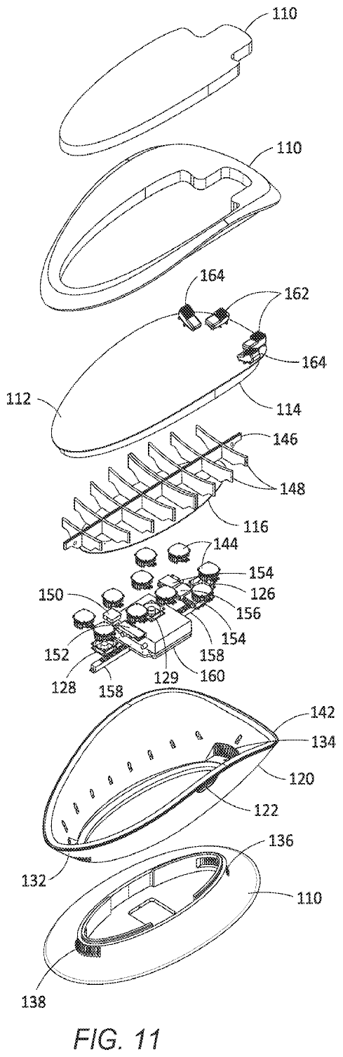

FIG. 1 illustrates a perspective view of a therapy bed 100. The therapy bed 100 may be used to provide vibrational and sound therapy to a user. The therapy bed 100 may comprise a bed cushion 110, a frame 120, a base 130. The therapy bed 100 can comprise any suitable materials to support the purposes described herein. For example, the therapy bed 100 may comprise materials suitable to support a wide range of users (e.g., varying weights) while maintaining an exterior that promotes a pleasant feel and aesthetic look. The bed cushion 110 may comprise a resiliently compressible material that is configured to support a user. For example, the bed cushion 110 may comprise a cushion, a foam material (e.g., memory foam), and/or the like. In some embodiments, the bed cushion 110 may comprise more than one material. In some embodiments, the bed cushion 110 may include a filling material or no filling material. In some embodiments, the bed cushion 110 may comprise an exterior including, for example, leather, plastic, composite, and/or the like. Generally, the bed cushion 110 is designed to allow a user to lie comfortably during a therapy session while still maintaining its shape and look after repeated use. In use, a user may lie on the bed cushion 110 with their head positioned near the back of the therapy bed 100 and their feet positioned near the front of the therapy bed 100. The therapy bed 100 is shaped to support a large range of adult users with varying heights, weights, and body types such that a user's entire body is contained within the bed cushion 110. The therapy bed 100 may have a length (e.g., from the front side to the back side along a center line) longer than 5 feet, longer than 6 feet, longer than 7 feet, longer than 8 feet, and/or the like. Similarly, the therapy bed 100 may have a width (e.g., from the left side to the right side along a center line) wider than 2 feet, 3 feet, 4 feet, 5 feet, 6 feet, 7 feet, 8 feet, and/or the like. In some embodiments, the bed cushion 110 may have a thickness such that in use, the user is entirely supported by the bed cushion 110 and compression of the bed cushion 110 does not result in the user having direct contact with the bed cushion support 112 (e.g., as shown in FIG. 11). For example, the bed cushion 110 may be equal to or greater than ½ inch, 1 inch, 2 inches, 3 inches, 4 inches, 6 inches, 12 inches, and/or the like. Also, in some embodiments, there may be no bed cushion and the cushion support 112 may be the only support provided. In some embodiments, the bed cushion 110 may be of marginal thickness and to act as a cover to the cushion support. In some embodiments, the bed cushion 110, at any thickness, can provide a visual cover (e.g., to look more visually pleasing, to match a color scheme, to provide customizable options) and/or to provide a texture or feel over the surface of the bed cushion support (e.g., to increase friction, softness, or the like). In some embodiments, the bed cushion 110 may comprise a material that can be cleaned easily in between uses. For example, when the therapy bed 100 is used by multiple different users, it may be desirable to clean or wipe the bed cushion 110 in between uses by users.

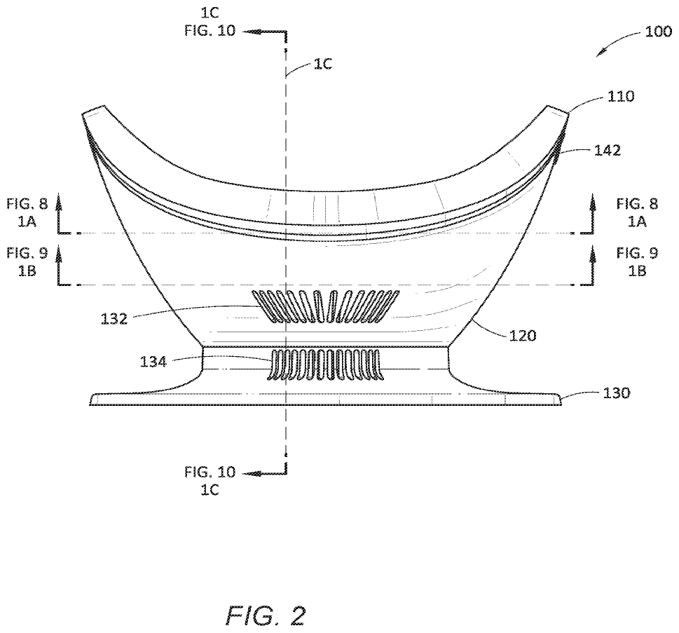

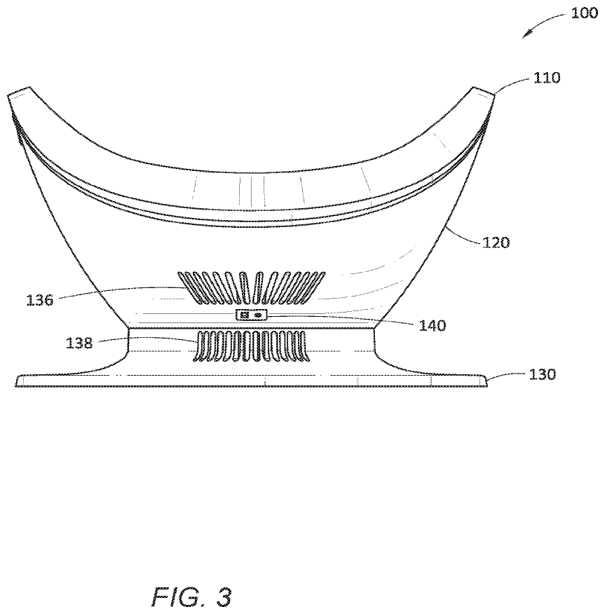

The frame 120 is configured to support and/or reinforce a specific shape of the bed cushion 110. In some embodiments, the frame 120 is designed to promote a pleasant feel for a user and may be designed to promote a calming effect on a user, while still sufficiently supporting the overall structure of the therapy bed 100. For example, the frame 120 may be rigid to support a wide range of users. As shown in FIGS. 2 and 3, the frame 120 may have a roughly curved/cocoon shape, with two wing portions. For example, a user lying flat on the bed cushion 110 may have a first wing portion on their left side and a second wing portion on their right side. The curved wings that a user lies between may be configured to help support and calm the user's nervous system by providing a safe sense of security and protection once the user lays down in the cocoon shaped therapy bed 100. For example, the cocoon shape may result in a user experiencing a deep subconscious state from their time in the womb, being cradled as a baby, being held as a child, and even as adults feeling the warm embrace of a loving hug, allowing the user's body to feel safe. Further, the cocoon shape may help a user's nervous system switch off the fight or flight mode from a sympathetic state (stress/adrenals) to a parasympathetic state (relaxed). The frame 120 may comprise any suitable material to support the load of a user. For example, the frame 120 may comprise a plastic, metal, wood, and/or the like material. In some embodiments, the frame 120 may have a consistent cocoon shape. In some embodiments, the frame 120 may be configured to move from a first position to a second position which changes the shape of the therapy bed 100. For example, the frame 120 may comprise two automated wing portions that extend between a first flat position where the bed cushion 110 is approximately flat, similar to a traditional bed, and second cocoon position, where the bed cushion 110 is curved, as illustrated in FIGS. 2 and 3. In some embodiments, the frame 120 wings may be configured to extend to different curves that may be customizable for a user. For example, a first user may input a first curved position and a second user may input a second curved position. In some embodiments, the therapy bed 100 may be configured to transition to the second cocoon position in response to detecting a user. For example, the therapy bed 100 may include sensors (e.g., one or more weight transducers) that determine when a user is on the therapy bed 100 and the control system may cause the wings of the frame 120 to transition from the flat position to the cocoon position. In some embodiments, the therapy bed 100 may be configured to determine who the user is based on the detected weight of the user. For example, users may have specific setting and profiles that are customizable, as described further herein. In some embodiments, the therapy bed 100 may have a default (e.g., when no user is present) flat shape. In some embodiments, the therapy bed 100 may have a default (e.g., when no user is present) cocoon shape. In some embodiments, the therapy bed 100 may change from a default state to a flat state to receive a user in response to detecting the presence of a user. For example, the therapy bed 100 may include one or more sensors to determine if a user is approaching the therapy bed 100.

The therapy bed 100 may include a light system 142. The light system 142 may extend around the frame 120 or another portion of the therapy bed 100. The light system may comprise one or more light emitting diodes (LED, OLED, etc.), compact fluorescent lamps (CFL), halogen lamps, incandescent bulbs, and/or the like. In some embodiments, the light system 142 may comprise a plurality of bulbs positioned beneath a diffuser to create an aura of light around the therapy bed 100. In some embodiments, the light system 142 may be configured to generate a plurality of different colors. For example, different colors may support and/or correspond to different emotional or spiritual states. Because color photons have individual wavelengths and frequency (e.g., are visually vibratory), a user's body may recognize the waveforms when exposed to the light system 142 and have a bodily or emotional response to the colored light. For example, users can perceive color due to the vibration or frequency attribute that a color comprises. So, users can experience a specific colored/visual aura and energy for their experience and time using the device. As described further herein, the colors may be selectable by a user (e.g., by an app associated with the therapy bed 100) and/or may be selected to relate to the vibrational and sound therapy a user is experiencing. In some embodiments, the light system 142 may generate colors that match emotions the user is experiencing (e.g., as determined by user inputs) and a user may be able to adjust the color via an app associated with the therapy bed 100 or manually on the therapy bed 100. For example, colors may match energies such as, for example, red matching high energy, blue matching calmer energy, and/or the like. In some embodiments, the colors produced by light system 142 may vary for the duration of the therapy. In some embodiments the colors produced by light system 142 may be consistent for the duration of the therapy. In some embodiments, the colors produced by the light system 142 may be selected by a machine learning algorithm/artificial intelligence as described further herein.

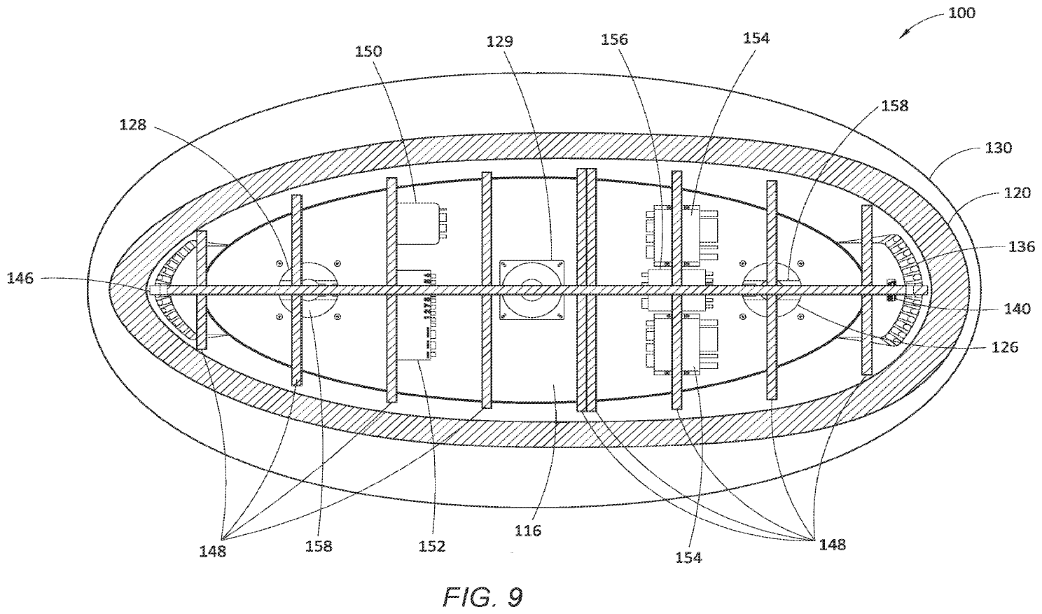

FIGS. 2 and 3 illustrate a front side view and a back side view respectively of the therapy bed 100. As shown, the therapy bed 100 is supported by a base 130 that is configured to distribute the weight of the therapy bed 100 over a large area. The base 130 may be configured to prevent significant vibration of the therapy bed 100 when in use. The base 130 may comprise any suitable material to support the uses described herein, and generally is designed to support a wide range of users. In some embodiments, one or both the frame 120 and the base 130 may include a plurality of vents, such as, for example, front frame vent 132, front base vent 134, back frame vent 136, back base vent 138. The plurality of vents may allow air to travel in a front to back direction or a back to front direction and may be part of an air-cooling system that include fans positioned within the frame (e.g., first fan 126, second fan 128, and/or third fan 129 shown in FIG. 9). The air-cooling system including the plurality of vents and fans may be used to cool electrical components and other internal components of the therapy bed 100.

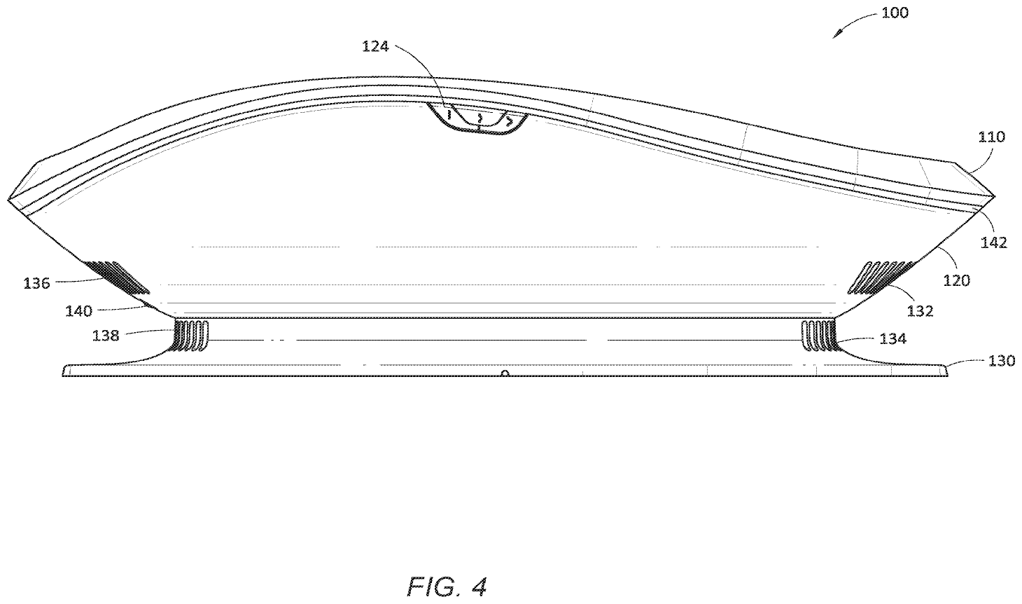

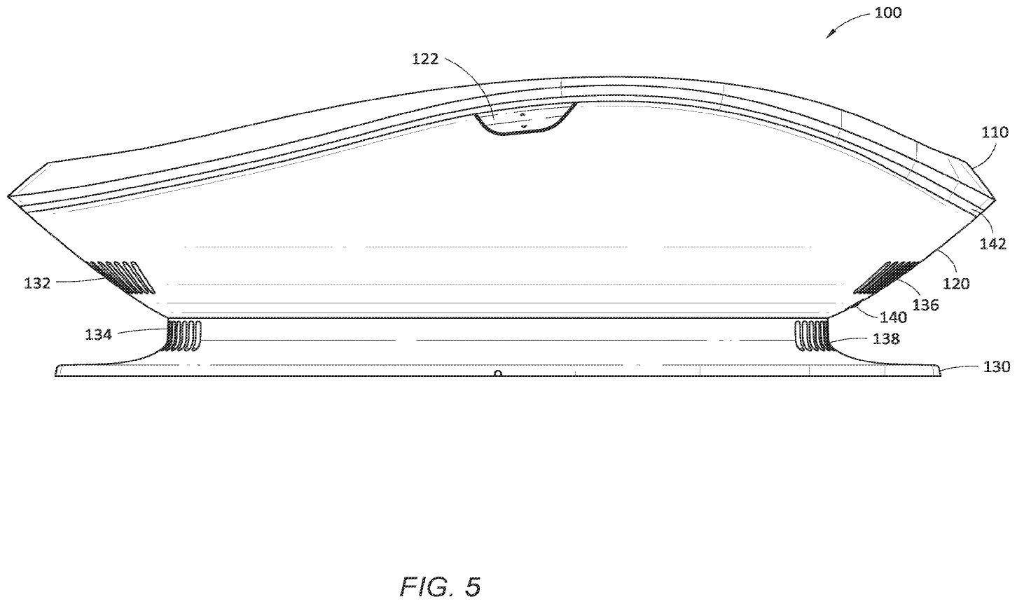

FIGS. 4 and 5 illustrate a left-side view and a right-side view respectively of the therapy bed 100. As shown, the therapy bed 100 can include one or more user control systems, such as, for example, left user control 124 and right user control 122. The left user control 124 and right user control 122 may be positioned on the frame 120 and may be located in a positioned such that a user lying down can reach each control with their left and right hands respectively, with minimal impact to their therapy experience. For example, a user may not be required to exit the therapy bed 100 to change the settings on the therapy bed 100. The two user controls 122, 124 may be used to control various functions and systems of the therapy bed 100. Example controls may include one or more control options related to the sound therapy, such as, for example, play, pause, skip, rewind, fast forward, volume increase, volume decrease, and/or the like. In some embodiments, controls may include control options related to the vibrational therapy such as, for example, increased vibration, decreased vibration, and/or the like. In some embodiments, controls may include control options related to the position of the system, such as, for example, to increase or decrease the position of the wings of the frame 120. In some embodiments, users may be able to control the vibroacoustic bass channels and audio music separately with built in volume/play/pause/skip buttons on the therapy bed 100 for quick access to fully customize the level of vibration and sound an individual user prefers. In some embodiments, the left user control 124 may control sounds and/or vibrational settings and the right user control 122 may control positional settings or vice-versa. In some embodiments, the user controls 122, 124 may comprise buttons that can be pushed or compressed. In some embodiments, the user controls 122, 124 may comprise a touch screen. In some embodiments, all the controls and settings associated with the therapy bed 100 may be controllable by a user device (e.g., user device 202). For example, a user may be able to modify position, sound, vibration, and/or the like via an app associated with the therapy bed 100 accessible through a user device. In some embodiments, the therapy bed 100 may include a headphone input and output system 140, that may be configured to receive a wired headphone set and may allow the user to experience the sound therapy through a wired headphone system. In some embodiments, the therapy bed 100 may be configured to support a wireless sound experience via wireless headphones (e.g., Bluetooth). For example, a user may be able to experience the sound therapy via wireless headphones which may provide benefits or reducing the noise impact on other users when multiple therapy beds 100 are in close proximity to each other.

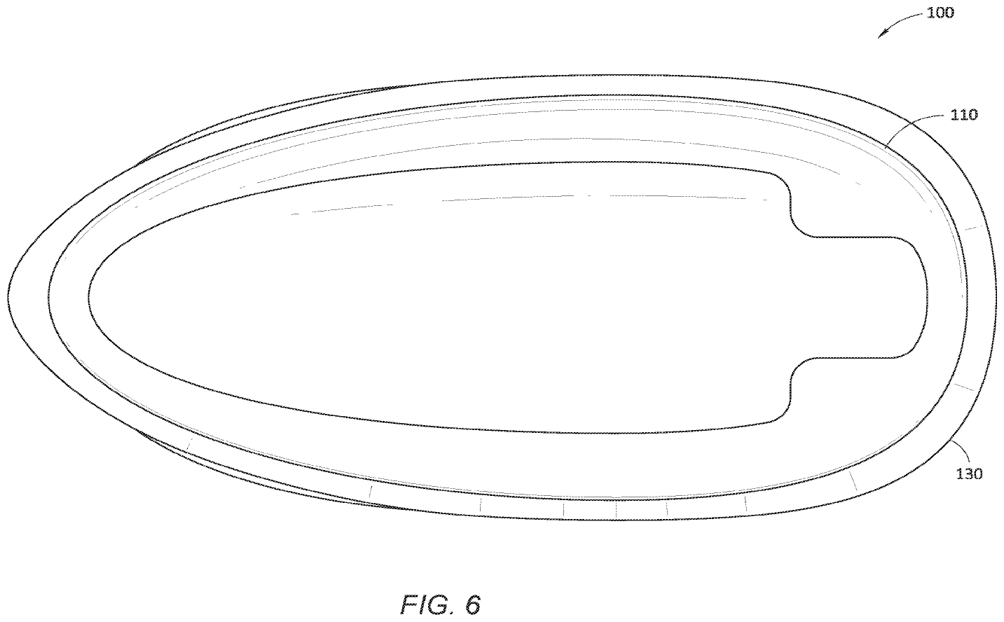

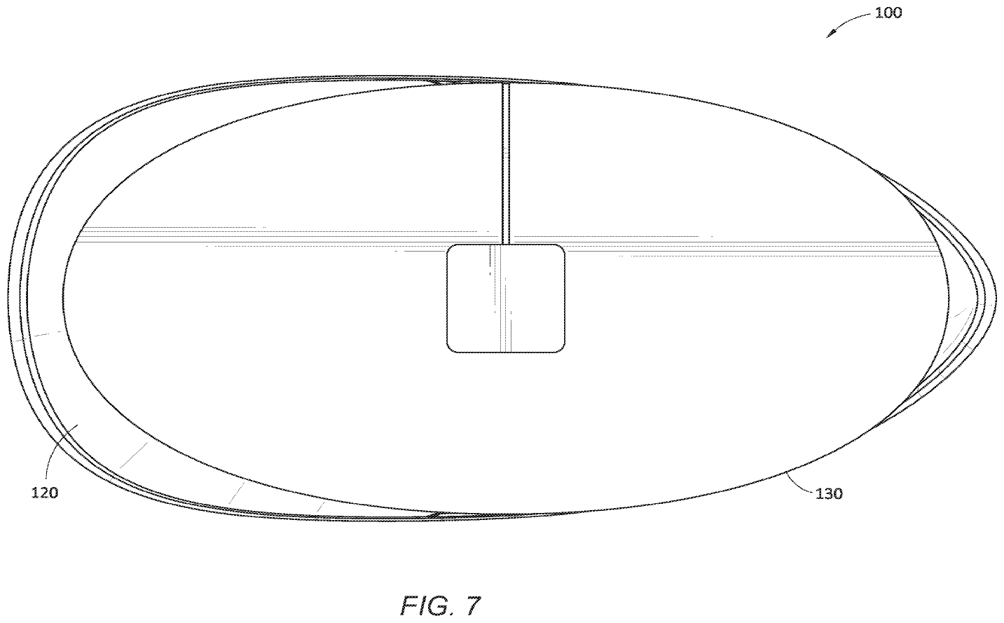

FIGS. 6 and 7 illustrate a top view and bottom view respectively of the therapy bed 100. As shown from these views, the therapy bed 100 has an approximately pod shaped design. In some embodiments, the therapy bed 100 may comprise a different shape, such as, for example, a circular shape, a rectangular shape, a polygonal shape, and/or the like.

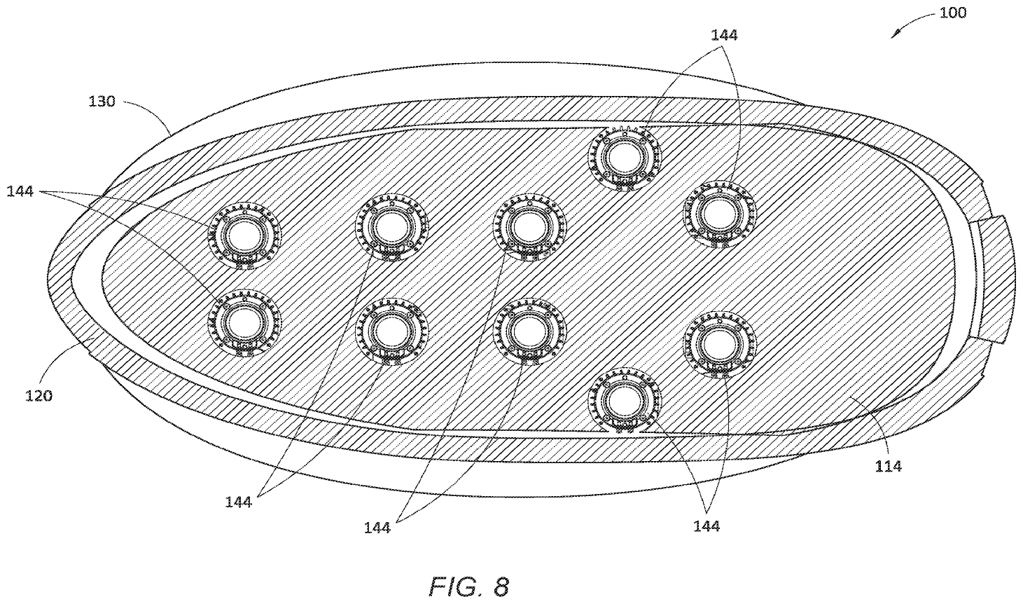

FIG. 8 illustrates a section view of the therapy bed 100 along the line 1A-1A in FIG. 2. As shown, the frame 120 may include a bed cushion backer 114. Positioned within the bed cushion backer 114 can be a plurality of transducers 144 (e.g., high powered bass transducers). While ten transducers 144 are illustrated, the therapy bed 100 may include any number of transducers 144 to support the vibrational therapy. For example, the therapy bed 100 may include 2, 4, 6, 8, 10, 12, 14, 16, 18, 20, 30, 40 and/or the like transducers 144. The transducers 144 are configured to produce vibrations such that, in use, a user feels the vibration and bass with their body while simultaneously hearing the music associated with the sound therapy. In some embodiments, the transducers 144 may be positioned to target specific portions of a user's body with vibrational therapy. For example, transducers 144 may be positioned beneath a user's legs, hips, backs, shoulders, and/or the like. As described further herein, the combination of a user's body feeling the music through their body via the transducers 144 and hearing the music through their ears (e.g., via speakers or headphones), creates an out of body experience that may resemble a meditative state and may allow a user to achieve a higher state of consciousness. In some embodiments, the bed cushion backer 114 and/or other components of the therapy bed 100 may be configured to dampen the vibrations produced by the plurality of transducers 144 such that minimal vibration is transmitted outside of the therapy bed 100. For example, a user may experience vibrational therapy, but external persons may not feel the vibrations. Dampening the vibration of the therapy bed 100 may provide benefits of allowing the therapy bed 100 to be used on elevated floors of buildings and may allow multiple therapy beds 100 to be in operation in a room.

In some embodiments, the audio delivered by the system may be split into multiple channels and diverted to different areas of the system. For example, some channels (e.g., low frequency) may be directed to the transducers 144 to cause or contribute to the vibrations. In another example, some channels (e.g., mid frequency, high frequency, and/or the like) may be diverted to the speakers 162 and 164 of headphones. In some cases, splitting the audio contributes to the combination of the user's body feeling the music (e.g., the low frequency channel) while also hearing the music (e.g., the mid/high frequency channel). In some embodiments, the way the audio is distributed may vary depending on whether a user is listening to the audio via headphones or via the speakers (e.g., speakers 162 and 164). For example, when using headphones, all the audio channels may be diverted to the headphones. In another example, when using the speakers 162 and 164, only a portion of the audio channels may be directed to the speakers 162 and 164. In some embodiments, all of the audio channels may be directed to the speakers 162 and 164.

In some embodiments, the therapy bed 100 includes a multi-channel bass amplifier system that allows a user to have immersive experiences by turning on the bass in different zones at different times. For example, the transducers 144 may be positioned in one or more groupings, such as, for example, rows, quadrants, and/or the like. A user may be able to select a vibrational therapy that has different transducers 144 and/or different groupings of transducers 144 producing different types and quantities of vibrations at different times. For example, one vibrational/sound therapy may include a rolling waves of bass that turn different transducers 144 on one by one starting from transducers 144 positioned closest to the user's feet and continuing through each transducer 144 working towards a user's head. As the vibrational/sound therapy continues, the wave pattern of vibrations may repeat. This rhythmic wave of bass running through a user's entire body like a wave, may create an experience of total immersion and transcendence. In some embodiments, the vibrational/sound therapy may include transducers 144 being activated in patterns related to the sounds the user is experiencing. For example, different transducers 144 may be activated on the left and right, top and bottom, and/or the like sides of a user at various times. In some embodiments, users will experience a range of different vibrations during a therapy session. In some embodiments, the vibrational therapy may include the transducers 144 being activated from the farthest outside location on a user's sides and working towards the middle. For example, the transducers 144 closest to the outside of the user's arms may activate followed by the next closest transducer(s) 144 to the middle of the bed cushion 110 and so on. In another example, the pattern may be switched, with transducers 144 closest to the middle of the bed cushion 110 being activated and transducer's 144 closer to the edge of the bed cushion 110 being progressively activated. In some embodiments, the transducers 144 may be activated moving from the front to the back of the therapy bed 100 or vice-versa. In some embodiments, the transducers 144 may be activated to create a diagonal pattern. In some embodiments, the transducers 144 may be activated in a random order. It is recognized that while a few examples of different patterns have been provided, the transducers 144 can be activated in any order or groupings to create a desired vibrational therapy routine.

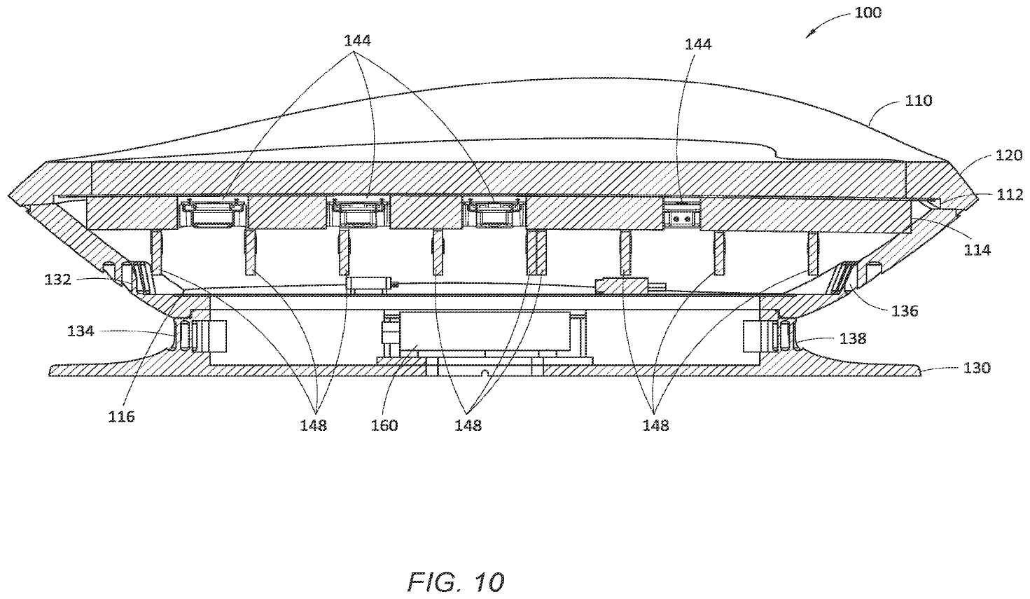

FIG. 9 illustrates a section view of the therapy bed 100 along the line 1B-1B in FIG. 2. FIG. 9 further illustrates some of the internal support structures and electrical/control systems of the therapy bed 100. As shown, the therapy bed 100 can include a plurality of internal ribs to support the bed cushion 110 and a user in use. In some embodiments, the therapy bed 100 may include one or more longitudinal ribs such as, for example, central bed frame rib 146. In some embodiments, the therapy bed 100 may include one or more latitudinal ribs such as, for example, bed frame ribs 148. While a certain number of central bed frame ribs 146 and bed frame ribs 148 are illustrated, it is recognized that the therapy bed 100 can include any number of central bed frame rib(s) 146 and bed frame ribs 148 to sufficiently support a wide range of users. For example, the therapy bed 100 may include 1, 2, 3, 4, 5, 8, 10, and/or the like central bed frame ribs 146. Similarly, the therapy bed 100 may include 1, 2, 3, 4, 5, 8, 10, 12, 15, 20, 30, and/or the like bed frame ribs 148. In some embodiments, the central bed frame rib 146 and the bed frame ribs 148 may be supported by a frame shelf 116. As shown more clearly in FIG. 10, the frame shelf 116 may be elevated above the base 130 and may support additional components of the therapy bed 100.

With continued reference to FIG. 9, the therapy bed 100 may include one or more fans associated with a cooling system. For example, the therapy bed 100 may include a first fan 126, a second fan 128, and/or a third fan 129. In some embodiments, the therapy bed 100 may include more or less than three fans. For example, the therapy bed 100 may include, 1, 2, 3, 4, 6, 8, 10, 15, 20, and/or the like fans. In some embodiments, the fans 126, 128, and/or 129 may be positioned on the frame shelf 116. In some embodiments, the fans 126, 128, and/or 129 may be positioned on or near the base 130. The fans 126, 128, and/or 129 may be configured to produce a flow of air that cools the internal electrical components and/or the transducers 144 of the therapy bed 100. The flow of air may enter the therapy bed 100 via the front frame vent 132 and/or the front base vent 134 and exit the therapy bed 100 via the back frame vent 136 and the back base vent 138 or vice versa.

In some embodiments, the therapy bed 100 may include one or more receivers 150 that may be positioned on the frame shelf 116. The one or more receivers 150 may be wireless and may be a High-Definition Multimedia Interface (“HDMI”) receiver. The one or more receivers 150 may be used in conjunction with the audio system of the therapy bed 100. In some embodiments, the therapy bed 100 may include a convertor 152 that may be positioned on the frame shelf 116. The convertor 152 may comprise a HDMI direct to analog convertor and may be used in conjunction with the audio system of the therapy bed 100. In some embodiments, the therapy bed 100 may include one or more amplifiers 154 that may be positioned on the frame shelf 116. The one or more amplifiers 154 may comprise mini-amplifiers and may be used in conjunction with the audio system of the therapy bed 100. In some embodiments, the therapy bed 100 may include one or more headphone amplifiers 156 that may be positioned on the frame shelf 116. For example, the one or more headphone amplifiers 156 may be configured to amply the sound a user experiences via the headphones and/or improve the sound quality when a user is using headphones. In some embodiments, the therapy bed 100 may include one or more power strips 158 which may be positioned on the base 130. The one or more powers strips 158 may be configured to connect to a power source (e.g., an outlet) where the therapy bed 100 in placed and may provide power to various system components (e.g., transducers 144, speakers 162 and 164, one or more receivers 150, convertor 152, one or more amplifiers 154, one or more headphone amplifiers 156, power amplifier 160, and/or the like).

FIG. 10 illustrates a section view of the therapy bed 100 along the line 1C-1C in FIG. 2. FIG. 10 illustrates example vertical positions of various components within the therapy bed 100. As shown, the therapy bed 100 may include a power amplifier 160. The power amplifier 160 may be configured to increase the magnitude of the power of the input signal. In some embodiments, the power amplifier 160 may be positioned on the base 130

FIG. 11 illustrates an exploded perspective view of the therapy bed 100. As shown, the therapy bed 100 can include a plurality of speakers, such as, for example, top speakers 162 and side speakers 164. The speakers 162 and 164 may be on the bed cushion support 112 and may be located near the back of the therapy bed 100 such that the speakers surround a user's head while a user undergoes therapy on the therapy bed 100. The speakers 162 and 164 may be surround sound speakers and may be positioned around a user's crown/head for an immersive spatial sound experience. The combination of a user's body feeling the music and a user's ears hearing the music, creates an outer body experience that takes meditation to another level, allowing a user to achieve a higher state of consciousness. In some embodiments, including the embodiment illustrated, the therapy bed 100 includes a left and right top speaker 162 and a left-side and right-side speaker 164. In some embodiments, the therapy bed 100 may include, for example. 2, 4, 6, 8, 10, and/or the like top speakers 162. In some embodiments, the therapy bed 100 may include, for example. 2, 4, 6, 8, 10, and/or the like side speakers 164. In some embodiments, the speaks 162 and 164 may be positioned on angled brackets configured to direct the speakers and output audio towards a user's ears in a manner to create a disconnect from the sound the user is hearing and the vibrations the user is experiencing. For example, the side speakers 164 may have an angle relative to the bed cushion support 112 greater than 5, 10, 15, 20, 25, 30, 35, 40, 45, 50, 55, 60, 65, 70, 75, 80, 85, and/or the like degrees. Similarly, top speakers 162 may have an angle relative to the bed cushion support 112 greater than 5, 10, 15, 20, 25, 30, 35, 40, 45, 50, 55, 60, 65, 70, 75, 80, 85, and/or the like degrees. In some embodiments, the brackets for the top speakers 162 may be configured to direct the audio so that the audio is elevated above the back of a user's head to, for example, promote an immersive audio experience. For example, the brackets for the top speakers 162 may direct the top speakers 162 at or above the ear-level of a user in a reclined position. In some embodiments, the brackets for the side speakers 164 may be configured to direct the audio from the middle of the speaker towards a user's ear. For example, the brackets for the side speakers 164 may direct the side speakers 164 at or near the ear-level of a user in a reclined position. While using the therapy bed 100, users can select a wide range of binaural beats and frequencies that support improved brainwave synchronization for both sides of the brain's hemispheres which may result in better neuro communication of thoughts, improved brain performance, and/or optimizing mental and emotional health. In some embodiments, the bed cushion 110 may include one or more perforations (e.g., 2, 4, 6, 8, 10, 20, 30, 50, 100, and/or the like preformation) to improve the sound experience of a user. In some embodiments, users may be able to choose to receive the audio related to the sound therapy via the internal speakers 162 and 164 or external wired or wireless headphones. Headphone may provide, for example, a more personalized and deeper experience in sound meditation.

In some embodiments, the therapy bed 100 may include one or more heat systems (e.g., infrared heat strips). The heat system may be positioned within the therapy bed 100 to provide the user with an increased temperature. For example, a heat strip may be positioned along the longitudinal center line of the therapy bed 100 to provide heat to a user's spine.

In some embodiments, the therapy bed 100 may be configured to support more than one user at a time. For example, the therapy bed 100 may be used for a couple's therapy session. Multiple users may lie side-by-side and may experience vibrational and sound therapy. In a multiple user embodiment, the therapy bed 100 may be larger than a single user therapy bed 100 or the therapy bed 100 may maintain the flat position for the therapy. In a multiple user embodiment, headphones may provide benefits of allowing users to experience both left and right side audio.

In some embodiments, a therapy session may include one or both sound therapy and vibrational therapy. In some embodiments, a therapy session may be between 1 minutes and 60 minutes. For example, a therapy session may be longer than 5 minutes, 10 minutes, 15 minutes, 30 minutes, 45 minutes, and/or the like. In some embodiments, aspects including length, vibrational intensity, audio volume, and/or the like may be customizable for an individual user.

In some embodiments, a user may be able to control the therapy bed 100 using manual controls included in the therapy bed 100, such as, for example, right user control 122 and left user control 124. In some embodiments, a user may be able to control the therapy bed 100 using an app associated with the therapy bed 100. In some embodiments, the therapy bed 100 may be used in conjunction with an additional external or internal device to provide visual imagery associated with the sound and vibrational therapy. For example, as described further herein, every emotion in your mind/body, generates a specific frequency to that emotion. The therapy bed 100 may allow a user to choose from a vast list of music frequencies to not only help enhance a user's mood and support a healthy emotional foundation, but also allow a user to select music based on what emotion they would like to connect with at a deeper level. Based on the desired user sound input, an associated visual output may be presented to a user via virtual reality, augmented reality, and/or the like.

### IV. Therapy Environment

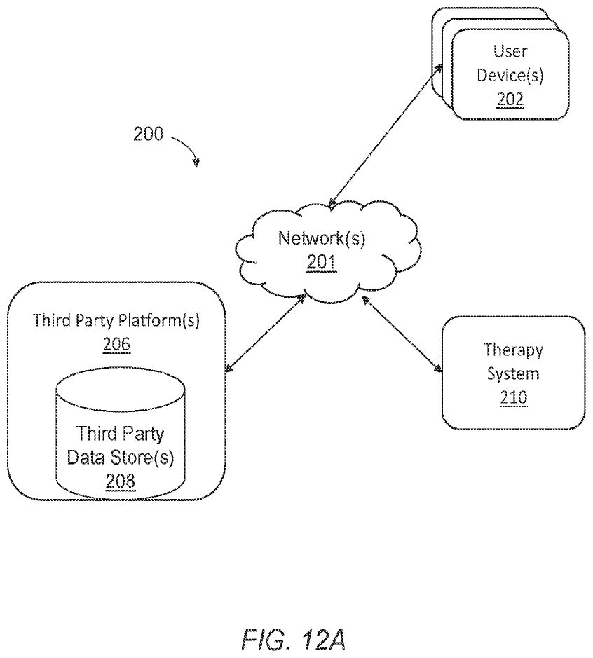

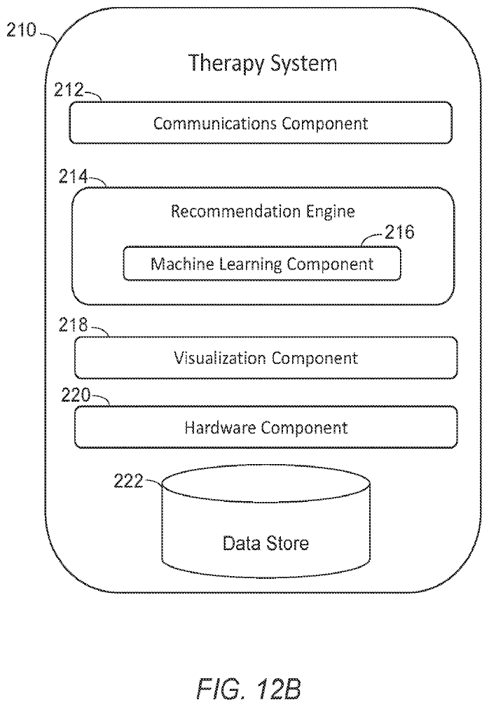

FIG. 12A is an example overall system diagram illustrating an embodiment of a therapy environment 200 for providing vibrational therapy, sound therapy, and/or other services to users using a therapy system 210. The environment 200 can include user device(s) 202 and third-party platform(s) 206 in communication over network 201 with therapy system 210. Therapy system 210 may include one or more subsystems and/or subcomponents. Embodiments of therapy system 210 will be further described with reference to FIG. 12B.

a. User Device(s)

In some embodiments, the user device(s) 202 may be a personal computer, a laptop computer, a smart phone, a tablet, smart watch, and/or the like, which can be used by a user to access a therapy system 210 over network 201. A user may access therapy system 210 to find coach using the platform, to communicate with a coach, view information (e.g., routine, historical data, assessment data etc.) related to their profile on the platform and/or the like. In some embodiments, a one or more user devices 202 can access the therapy system 210 in addition to, or instead of, accessing the therapy system 210 physically in person.

b. Third Party Platform(s)

In some embodiment, one or more third-party platform(s) 206 may be in communication with therapy system 210 over network 201. The third-party platforms 206 may comprise one database or multiple databases. For example, there may be a separate database corresponding to each third-party or data from multiple third parties may be stored using virtual partitions or access privileges to prevent the sharing of data among third parties. The third-party platforms 206 may be controlled by a database management system. The third-party platforms 206 may be configured to store data associated with recommendation engine 214 and/or other elements associated with the therapy system 210 as describe further herein. In some embodiments, the therapy system 210 may communicate directly with third-party platforms 206 over network 201 (e.g., via one or more APIs). A third party may be any third party with information that can be utilized by the therapy system 210. For example, a third party may be a healthcare provider (e.g., with medical information about a user, diagnostic information, and/or the like), sound therapy platform, an artist (e.g., who provides music to the system for sound therapy), and/or the like.

i. Third Party Data Store(s)

In some embodiments, the third-party platforms 206 may include, one or more third party data store(s) 208. The third-party data store(s) 208 may be configured to store data associated with one or more third-party platforms 206. For example, as described above, third party data store(s) 208 may store data related to medical information that can be accessed using, for example, the recommendation engine 214.

c. Therapy System

In some embodiments, a therapy system 210 may communicate with one or more devices (for example, user device 202) over network 201 to facilitate selection or recommendation of sound and vibrational routine selection for users, music selection for users, assessments for users, and/or the like. The therapy system 210 is described further herein with reference to FIG. 12B.

### d. Network(s)

In some embodiments, the network 201 may comprise one or more networks, including, for example, a local area network (LAN), wide area network (WAN), and/or the Internet, for example, via a wired, wireless, or combination of wired and wireless, communication links. The network 201 can facilitate communication between the user devices 202, third-party platforms 206, and the therapy system 210.

While FIG. 12A shows an example number of systems in communication with network 201, it is recognized that in some embodiments, multiple user devices 202, and multiple third third-party platforms 206 may be in communication with network 201 and the therapy system 210. Further, “multiple” can include, for example, tens, hundreds, thousands, or millions, of systems in communication with the therapy system 210. The devices in communication with therapy system 210 (for example, user devices 202) can each include one or more databases and/or parameters. The databases can include data associated with communications conducted by a user. It is recognized that the database may be stored in whole or in part on site in a facility or in one or more cloud storage locations.

### V. Therapy System

FIG. 12B illustrates an embodiment of the therapy system 210 and platform subcomponents. Therapy system 210 may include one or more of the following subcomponents: communications component 212, recommendation engine 214, visualization component 218, hardware component 220, and data store 222. The therapy system 210 may include one or more of each subcomponent for each service offered by the platform. For example, there may be a recommendation engine 214 that is utilized for sound therapy selection, a recommendation engine 214 that is utilized for vibrational therapy selection, a recommendation engine 214 that is utilized for visual therapy selection, and/or the like It is recognized that there are other embodiments of the therapy system 210 which may exclude features of the example therapy system 210 and/or may include additional features. As such, some of the processes and/or modules discussed herein may be combined, separated into sub-parts, and/or rearranged to run in a different order and/or in parallel. In addition, in some embodiments, different blocks may execute on various components of the therapy system 210.

a. Communication Component

In some embodiments, the communications component 212 may be configured to facilitate communication between the therapy system 210 and other systems and devices. For example, the communications component 212 may facilitate communication with user devices 202 and/or third-party platforms 206. In some embodiments, the communications component 212 may include one or more data input components and one or more data output components. The one or more data input components may be configured to receive and process various input data into the therapy system 210. The one or more data output components may be configured to process and format various data and results of the various analyses for access by other systems, such as the user devices 202 and/or third-party platforms 206.

In some embodiments, the therapy system 210 may be compatible with and can be used in conjunction with any combination of the embodiments, implementations, or features described in International Patent Publication No. WO 2022/251866 (the '866 publication), filed May 26, 2022, entitled “GENERATING RECOMMENDATIONS BY UTILIZING MACHINE LEARNING,” the disclosure of which is hereby incorporated herein by reference in its entirety for all purposes. Some or all of the features described herein can be used or otherwise combined together with any of the features described in the '866 publication. In one example, the communications component 212 can communicate with the systems described in the '866 publication.

b. Recommendation Engine 214

In some embodiments, recommendation engine 214 may be configured to determine, select, recommend, and/or match users with sound and vibrational therapy routines. Recommendation engine 214 may include one or more subcomponents, such as, for example, machine learning component 216, and/or the like. In some embodiments, recommendation engine 214 may include more or fewer subcomponents and in some embodiments, one subcomponent may perform the role of one or more other subcomponents. For example, the machine learning component can implement machine learning (“ML”) algorithms or artificial intelligence (“AI”) algorithms (generally collectively referred to herein as “AI/ML algorithms”, “AI/ML models”, or simply as “ML algorithms”, “ML models”, and/or the like) that may, for example, implement models that are executed by one or more processors.

i. Machine Learning Component 216

In some embodiments, features of the disclosed systems and methods may use one or more machine learning components to improve different aspects of the processes implemented by the system. For example, the machine learning component may update different elements related to the user's interaction with the system described herein. The machine learning component may include one or more machine learning systems/models, such as, for example, machine learning, artificial intelligence, neural networks, decision trees, and/or the like. For example, the machine learning component can implement machine learning (“ML”) algorithms or artificial intelligence (“AI”) algorithms that may, for example, implement models that are executed by one or more processors. Having an AI/ML model to facilitate user assessments and customized training can provide significant improvements as compared to conventional systems because weighting different factors/inputs may vary in unpredictable or surprising ways that the AI/ML model can be customized and trained to determine. In some embodiments, the machine learning component can use one or more machine learning algorithms to implement one or more models or parameter functions for the detections/identifications.

In some embodiments, a machine learning model can receive inputs it uses to train and/or apply the machine learning model to generate an output. In some embodiments, for example, and with respect to a particular user, inputs can include any and/or all user-provided or related information and data (e.g., interests, music, health conditions or issues, employment or employer information, demographic information, residency, third party data or access to third party accounts, marital information, age, sex, gender, visual or audio data, sensor data, or any other data provided by the user or on the user's behalf that may be pertinent to diagnosing a physical, mental, or emotional issue or customizing a therapy routine). For example, some professions require sitting all day, so certain therapies can focus on any issues that arise from sitting for extended periods of time. In some embodiments, the user's mood or emotional state (e.g., angry, sad, happy, or the like) may be used as inputs as well. In some embodiments, the user's online presence (e.g., social media and/or public records) or browsing habits may be used as inputs as well. In some embodiments, the inputs may be provided by APIs related to other products or applications. With respect to outputs from the machine learning model, for example, the machine learning model may output a determined list of ranked or recommended therapy routines (e.g., gentle to strong vibrational therapy, calming to energizing vibrational therapy, and/or the like), as compared to a particular user based on weighted inputs, where the weights are determined by the machine learning model during training. In another example, the machine learning model may output a determined list of ranked or recommend music for sound therapy as compared to a particular user based on weighted inputs, where the weights are determined by the machine learning model during training. In some embodiments, vibrational therapy routines and sound therapy routines are paired together and output as recommendation by the machine learning model. In another example, the machine learning model may output a determined list of ranked or recommend colors for delivering via the light system 142 for a therapy session as compared to a particular user based on weighted inputs, where the weights are determined by the machine learning model during training. In some embodiments, colors for delivering via the light system 142 during a therapy routine may be selected based on one or more of the selected vibrational therapy routines and/or sound therapy routines. For example, the color display may be paired with one or more of the selected vibrational therapy routines and/or sound therapy routines and output as a recommendation by the machine learning model. In some embodiments, the color display varies with the paired vibrational/sound therapy routine and changes over the course of a therapy session. For example, as the music and/or vibrational progress through a routine, the color displayed via the light system 142 may transition as well to match the music and vibrations. For example, where a routine (e.g., including vibrational and/or sound therapy) is selected to calm a user, the color may transition from a warm color (e.g., red, orange, yellow, and/or the like) to a cool color (e.g., green, blue, purple, and/or the like). In another example, where a routine (e.g., including vibrational and/or sound therapy) is selected to energize a user, the color may transition from a cool color to a warm color.

In some embodiments, the machine learning model can be trained based on annotated data comprising electronic information pertaining to successful and/or unsuccessful therapy routines. For example, a successful therapy routine may be a recommended therapy routine that a user completes 100% of the therapy routine. Also, for example, a successful therapy routine may be a therapy routine that user completes above a certain threshold (e.g., 70%, 80% of the therapy routine, or the like). Also, for example, a successful therapy routine may be a therapy routine that a user has indicated satisfaction (e.g., via on-screen feedback, through the user device 202, and/or the like). Also, for example, an unsuccessful therapy routine may be a therapy routine that a user has completed less than a certain threshold (e.g., 0%, 10%, 50% of the therapy routine, or the like). Also, for example, an unsuccessful therapy routine may be a therapy routine that a user has indicated dissatisfaction (e.g., via on-screen feedback, through the user device 202, and/or the like).

In some embodiments, a machine learning model can be further trained based on annotated data comprising electronic information pertaining to a magnitude of success or lack of success. For example, a length of time a user performs a therapy routine can be a factor used by the machine learning model during training or application of the model. For example, a user may perform the same therapy routine for longer than prescribed or multiple times in repetition indicating a higher magnitude of success than a user that may perform a portion of a therapy routine once. Another factor related to magnitude of success or lack of success, for example, can be an amount of improvement measured. For example, the machine learning model or machine learning component 216 can use data related to a user who has performed a therapy routine and where the user has improved significantly from the beginning to the end or upon repeating the same or similar therapy routine. A user that has shown improvement may indicate that the therapy routine is working and is therefore a successful recommendation based on the degree of improvement.

In some embodiments, a machine learning model can be trained based on annotated data comprising electronic information pertaining to successfully selecting music to improve a user's emotional state. For example, the machine learning model can be trained to correlate human emotional states to brain wave frequencies. For example, a successful music selection may be a music selection where a user identified an improved emotional state after listening to the music selection or after completing a therapy routine with the selected music. For example, a user may indicate improved emotional state (e.g., via on-screen feedback through the user device 202, and/or the like). Also, for example, an unsuccessful music selection may be a selection that a user indicated dissatisfaction. For example, dissatisfaction can include the user having a similar emotional state or an emotional state that is worse than that the user's emotional state prior to performing a recommended or selected therapy routine.

In some embodiments, a machine learning model can be further trained based on annotated data comprising electronic information pertaining to a magnitude of success or lack of success. For example, the magnitude of user identified improved emotional state may be an indication of success. For example, a user may indicate a significant improvement in emotional state indicating a higher magnitude of success than a user that may indicate a minor improvement in emotional state, no improvement in emotional state, decline in emotional state, and/or the like. Another factor related to magnitude of success or lack of success, for example, can be an amount of improved physical performance, balance, circulation, and/or inflammation of a user while completing a vibrational therapy routine while listening to selected music. For example, the machine learning model or machine learning component 216 can use data related to a user who has performed a vibrational therapy routine and where the user has improved significantly from previous completions of a therapy routine.

A number of different types of AI/ML algorithms and AI/ML models may be used by the therapy system 210. Further, these AI/ML models may be developed and/or trained using various methods. For example, certain embodiments herein may use a logistical regression model, decision trees, random forests, convolutional neural networks, deep networks, or others. However, other models are possible, such as a linear regression model, a discrete choice model, or a generalized linear model. The machine learning aspects can be configured to adaptively develop and update the models over time based on new input. For example, the models can be trained, retrained, or otherwise updated on a periodic basis as new received data is available to help keep the predictions in the model more accurate as the data is collected over time. Also, for example, the models can be trained, retrained, or otherwise updated based on configurations received from a user, admin, or other devices. Some non-limiting examples of machine learning algorithms that can be used to train, retrain, or otherwise update the models can include supervised and non-supervised machine learning algorithms, including regression algorithms (such as, for example, Ordinary Least Squares Regression), instance-based algorithms (such as, for example, Learning Vector Quantization), decision tree algorithms (such as, for example, classification and regression trees), Bayesian algorithms (such as, for example, Naive Bayes), clustering algorithms (such as, for example, k-means clustering), association rule learning algorithms (such as, for example, Apriori algorithms), artificial neural network algorithms (such as, for example, Perceptron), deep learning algorithms (such as, for example, Deep Boltzmann Machine), dimensionality reduction algorithms (such as, for example, Principal Component Analysis), ensemble algorithms (such as, for example, Stacked Generalization), support-vector machines, federated learning, and/or other machine learning algorithm. These machine learning algorithms may include any type of machine learning algorithms including hierarchical clustering algorithms and cluster analysis algorithms, such as a k-means algorithm. In some cases, the performing of the machine learning algorithms may include the use of an artificial neural network. By using machine-learning techniques, large amounts (such as terabytes or petabytes) of received data may be analyzed to generate or implement models with minimal, or with no, manual analysis or review by one or more people.

In some embodiments, supervised learning algorithms can build a mathematical model of a set of data that contains both the inputs and the desired outputs. For example, training data can be used, which comprises a set of training or labeled/annotated examples. Each training example has one or more inputs and the desired output, also known as a supervisory signal. In the mathematical model, for example, each training example is represented by an array or vector (e.g., a feature vector), and the training data is represented by a matrix. Through iterative optimization of an objective function, supervised learning algorithms can learn a function that can be used to predict the output associated with new inputs. An optimal function, for example, can allow the algorithm to correctly determine the output for inputs that were not a part of the training data. For instance, an algorithm that improves the accuracy of its outputs or predictions over time is said to have learned to perform that task. Types of supervised-learning algorithms may include, but are not limited to: active learning, classification, and regression. Classification algorithms, for example, are used when the outputs are restricted to a limited set of values. Regression algorithms, for example, are used when the outputs may have any numerical value within a range. As an example, for a classification algorithm that filters emails, the input would be an incoming email, and the output would be the name of the folder in which to file the email. In some embodiments, similarity learning, an area of supervised machine learning, is closely related to regression and classification, but the goal is to learn from examples using a similarity function that measures how similar or related two objects are. In some embodiments, similarity learning has applications in ranking, recommendation systems, visual identity tracking, face verification, and speaker verification.

In some embodiments, unsupervised learning algorithms can take a set of data that contains only inputs, and find structure in the data, like grouping or clustering of data points. For example, the algorithms can learn from test data that has not been labeled, classified, or categorized. Instead of responding to feedback, unsupervised learning algorithms can identify commonalities in the data and react based on the presence or absence of such commonalities in each new piece of data. In some embodiments, unsupervised learning encompasses summarizing and explaining data features. In some embodiments, cluster analysis is the assignment of a set of observations into subsets (e.g., clusters) so that observations within the same cluster are similar according to one or more predesignated criteria, while observations drawn from different clusters are dissimilar. In some cases, different clustering techniques can make different assumptions on the structure of the data, often defined by some similarity metric and evaluated, for example, by internal compactness, or the similarity between members of the same cluster, and separation, the difference between clusters. Other methods, for example, can be based on estimated density and graph connectivity.

In some embodiments, semi-supervised learning can be a combination of unsupervised learning (without any labeled training data) and supervised learning (with completely labeled training data). For example, some of the training examples may be missing training labels, and in some cases such training examples can produce a considerable improvement in learning accuracy as compared to supervised learning. In some embodiments, and in weakly supervised learning, the training labels can be noisy, limited, or imprecise; however, these labels are often cheaper to obtain, resulting in larger effective training sets.

In some embodiments, an area of machine learning is concerned with how software agents ought to take actions in an environment so as to maximize some notion of cumulative reward. In some embodiments, the environment is typically represented as a Markov decision process (MDP). In some embodiments, reinforcement learning algorithms use dynamic programming techniques. In some embodiments, reinforcement learning algorithms do not assume knowledge of an exact mathematical model of the MDP and are used when exact models are infeasible.

In addition to supervised learning algorithms, unsupervised learning algorithms, and semi-supervised learning, and in some embodiments, other types of machine learning methods can be implemented, such as: reinforcement learning (e.g., how software agents ought to take actions in an environment so as to maximize some notion of cumulative reward); dimensionality reduction (e.g., process of reducing the number of random variables under consideration by obtaining a set of principal variables); self-learning (e.g., learning with no external rewards and no external teacher advice); feature learning or representation learning (e.g., preserve information in their input but also transform it in a way that makes it useful); anomaly detection or outlier detection (e.g., identification of rare items, events or observations which raise suspicions by differing significantly from the majority of the data); association rules (e.g., discovering relationships between variables in large databases); and/or the like.

c. Visualization Component 218

In some embodiments, the visualization component 218 may be configured to generate user interfaces and display graphics for user devices 102. For example, the visualization component 218 may be used to present interactive graphical user interfaces including pain selection and emotional state selection, and/or the like. In some embodiments, the visualization component 218 may be configured to generate a visual therapy routine for a user as described above.

d. Hardware Component 220

In some embodiments, the hardware component 220 may be configured to interact with various hardware components described herein with reference to at least FIGS. 1-11. For example, the hardware component 220 may communicate with or include various device speakers, transducers, electrical components, and/or the like. In some embodiments, the hardware component 220 may be configured to activate various hardware components for use in the system. For example, the hardware component 220 may activate and control speakers 162, speakers 164, transducers 144, receivers 150, convertor 152, amplifiers 154, headphone amplifiers 156, power amplifier 160, and/or the like. In some embodiments, the hardware component 220 may control the movement of the wings of the frame 120. In some embodiments, hardware component 220 may control the various sensors described herein.

e. Data Store 222

In some embodiments, therapy system 210 may include a data component or individual data stores that may be configured to control and manage the storage of data within the therapy system 210. For example, data stores may respond to requests from various systems for accessing or updating the data stored within the therapy system 210. The data store 222 may comprise one data store or multiple data stores. For example, there may be a separate database corresponding to each user and each therapy bed 100, or data from multiple users and therapy beds 100 may be stored using virtual partitions or access privileges to prevent the sharing of data among users. The therapy system 210 may include a database management system.

### VI. User Environment

As described herein, a user may use the therapy bed 100 to undergo various vibrational and sound therapy routines. The user can use the therapy bed 100 not only to hear music, but also to feel music though their entire body at a cellular level. Use of the therapy bed 100 can result in a user experiencing enhanced mediation and recovery. While undergoing a therapy session, a user experiences the vibrations associated with the vibration therapy via the transducers 144 and experience the music associated with the sound therapy via the speakers 162 and speakers 164 and/or headphones. In some embodiments, the vibrations and music are related and are designed to achieve certain goals. For example, the vibrations and music may match a user's mood so a user can relate to what they're feeling on a physical and audible level. In another example, the vibration and music selection may be made to achieve a desired emotional state. In some embodiments, the vibrations and music are related to visual displays including external displays and light displays on the light system 142.

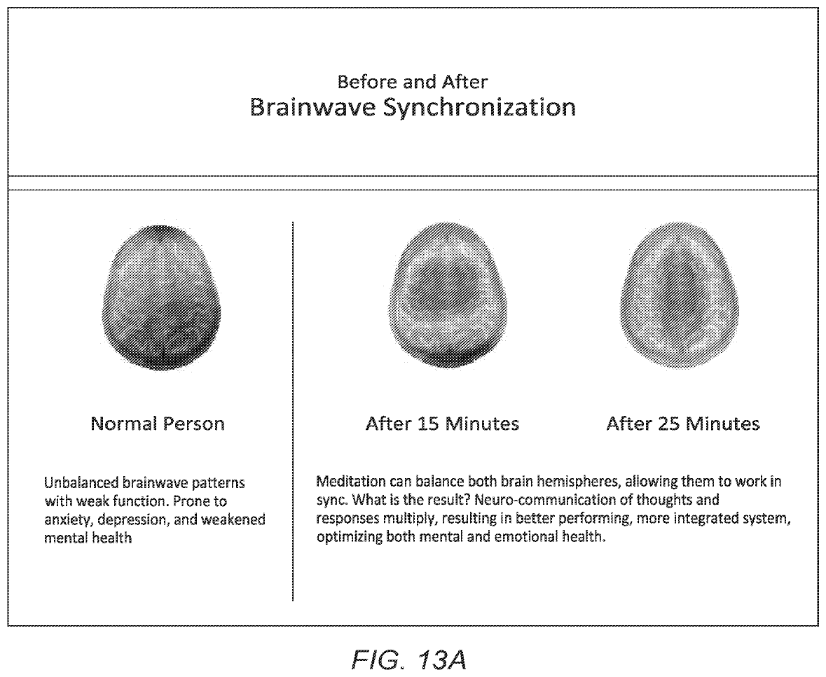

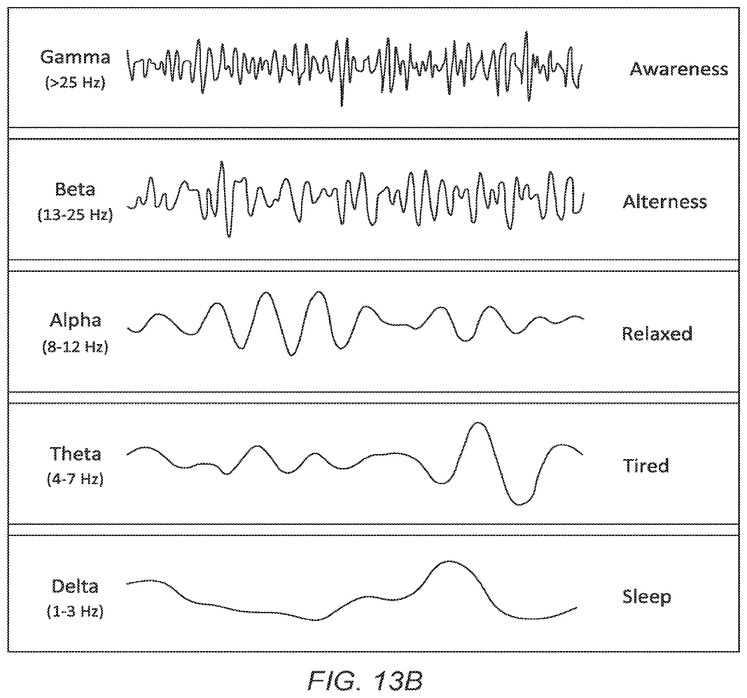

FIG. 13A illustrates an example brain before and after brainwave synchronization. In some embodiments, the therapy bed 100 may be configured to produce binaural beats to cause brainwave synchronization. For example, the sound therapy may include using slightly different tones in the audio delivered to the left and right ear of a user. Even though the tones are slightly different, the user's brain may perceive a single difference tone, resulting in improve brain synchronization. As shown in FIG. 13A, a normal person's brain may have unbalance brainwave patterns and weak function prior to receiving sound therapy. However, after a sound therapy session, similar to meditation, a user may experience better balance of the brain hemispheres, which may allow the brain hemispheres to work in sync.

FIG. 13B illustrates example binaural beats that may be delivered to a user using the therapy bed 100. In some embodiments, the therapy bed 100 may support sound therapy that encompasses a full range of brainwave frequencies: Gamma, Alpha, Beta, Delta, and Theta. As shown, each brainwave frequency may be associated with different emotional states such as, for example, awareness, alertness, relaxed, tired, sleep, and/or the like. By relating a desired or current emotion with a brainwave frequency, the therapy bed 100 may deliver a sound therapy routine to a user to help support physical and emotional healing and relaxation.

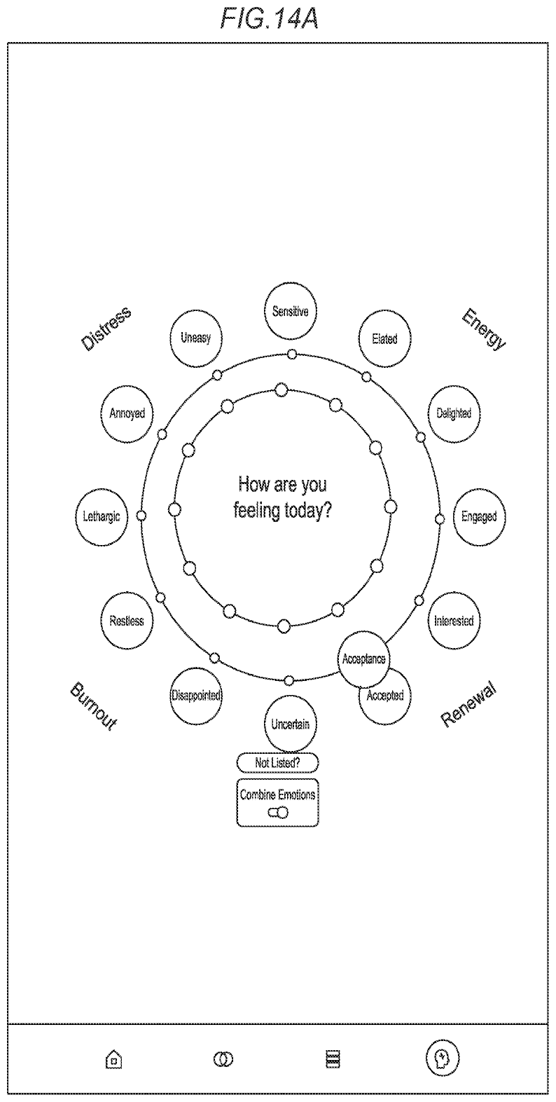

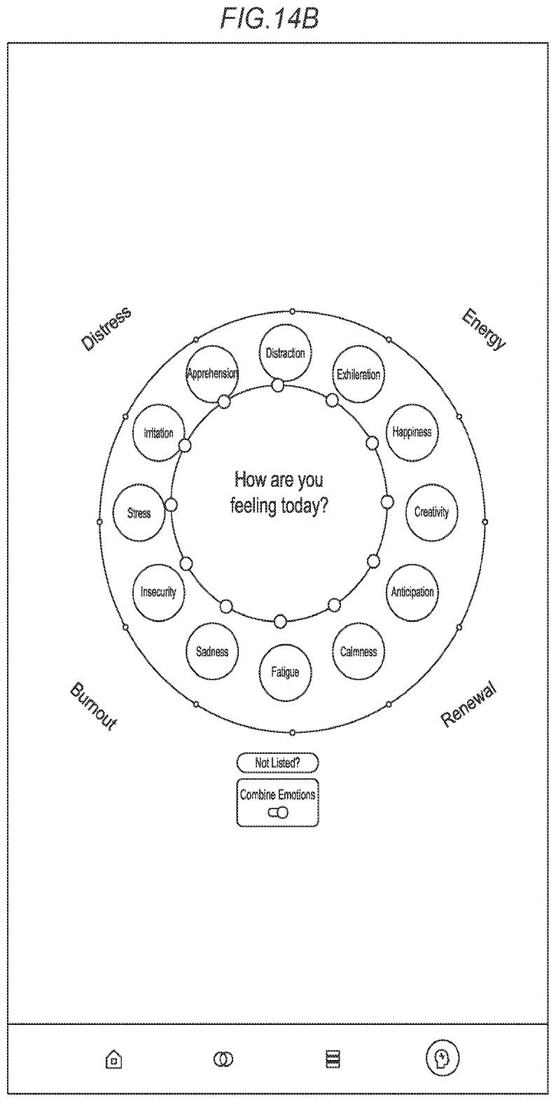

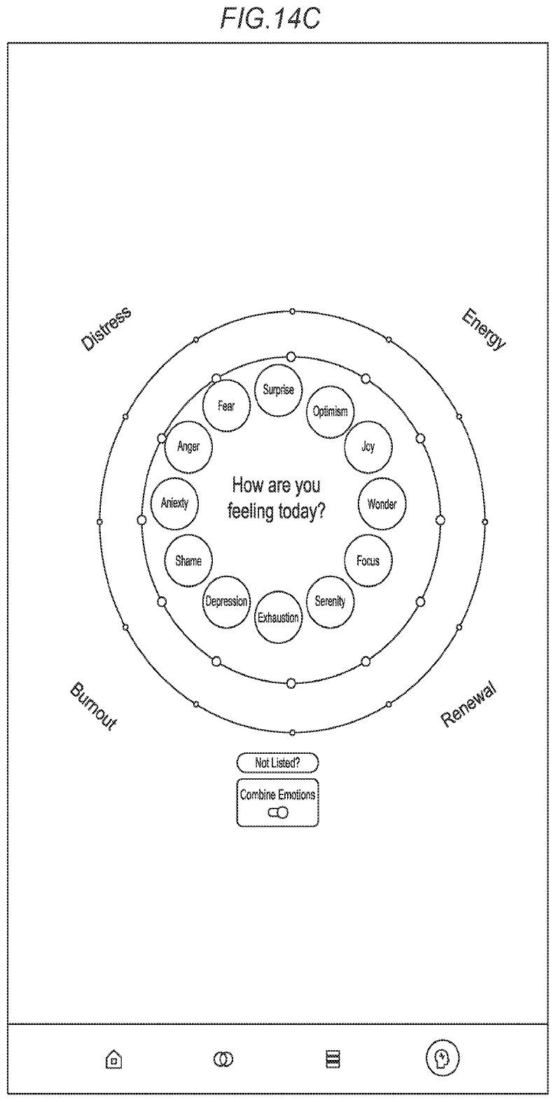

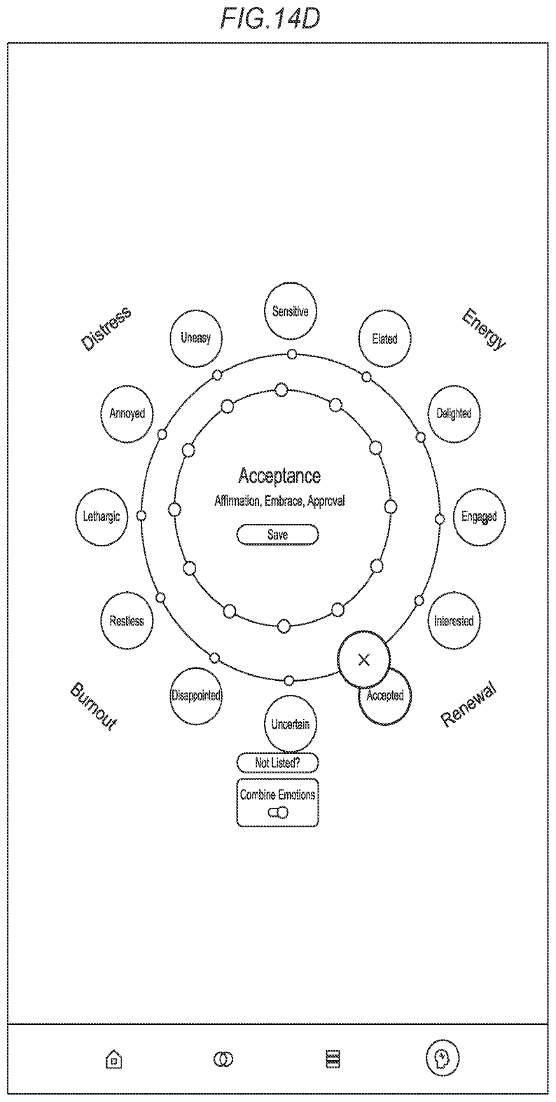

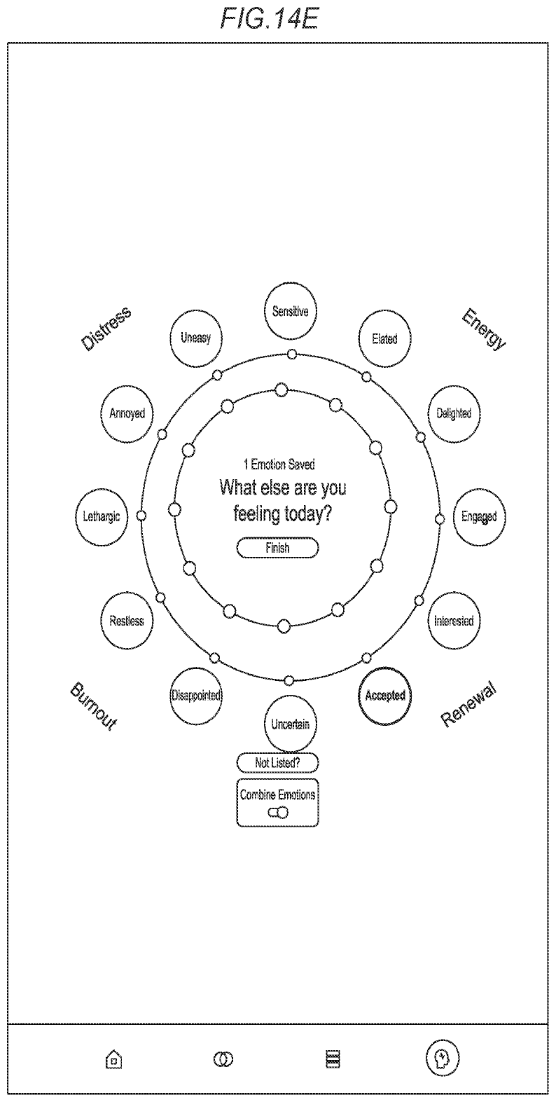

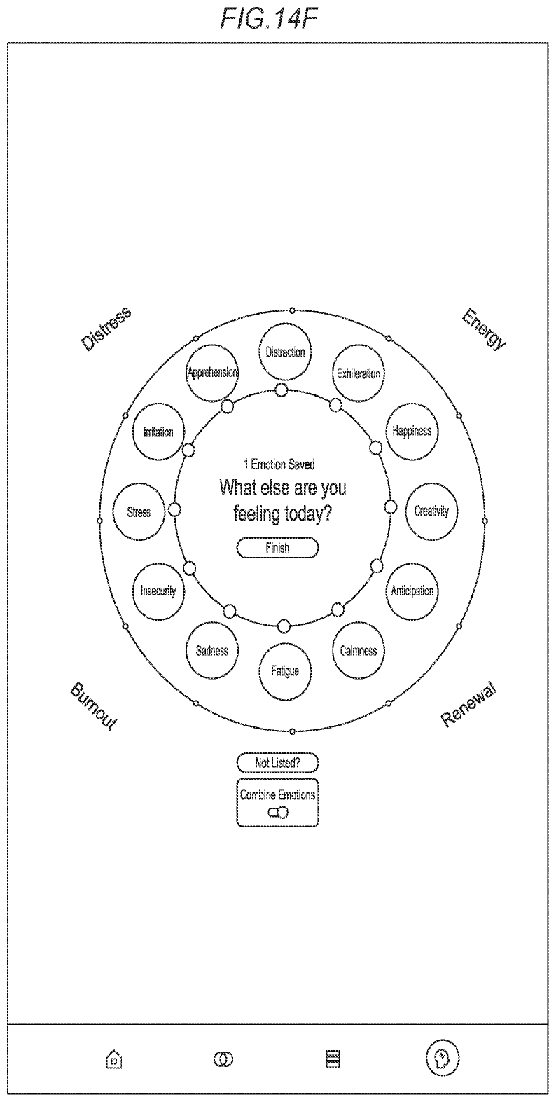

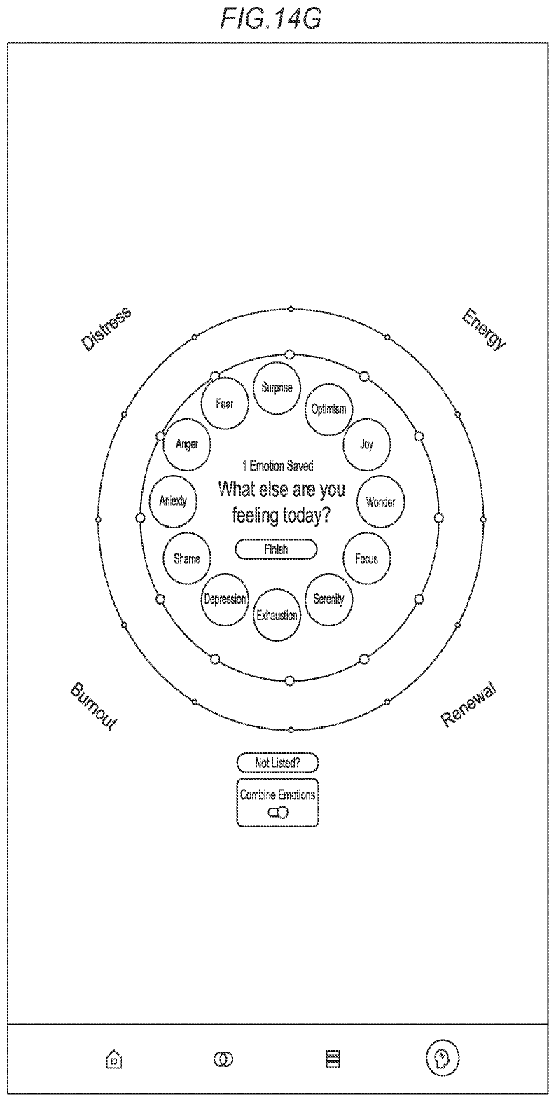

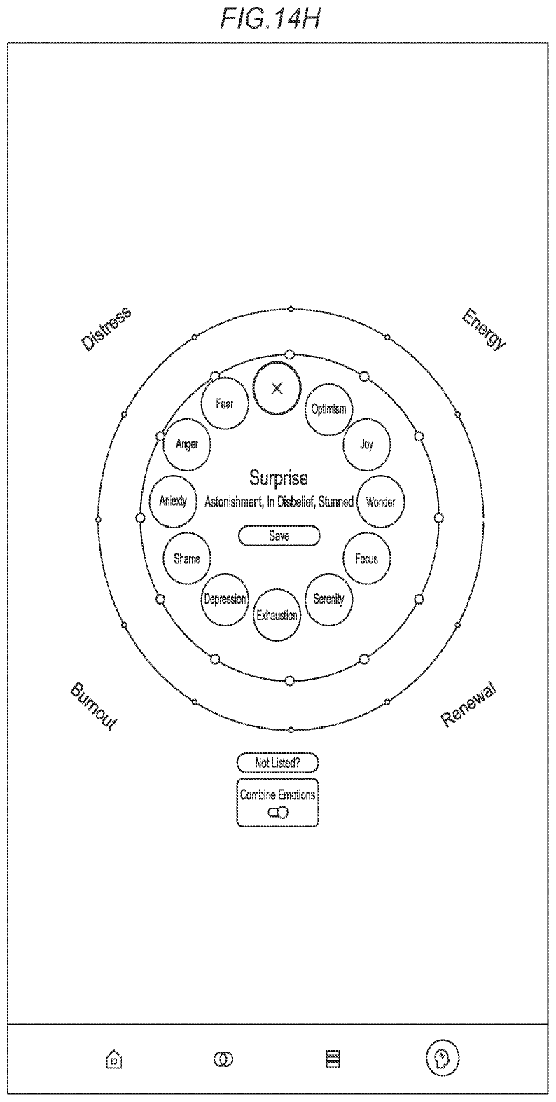

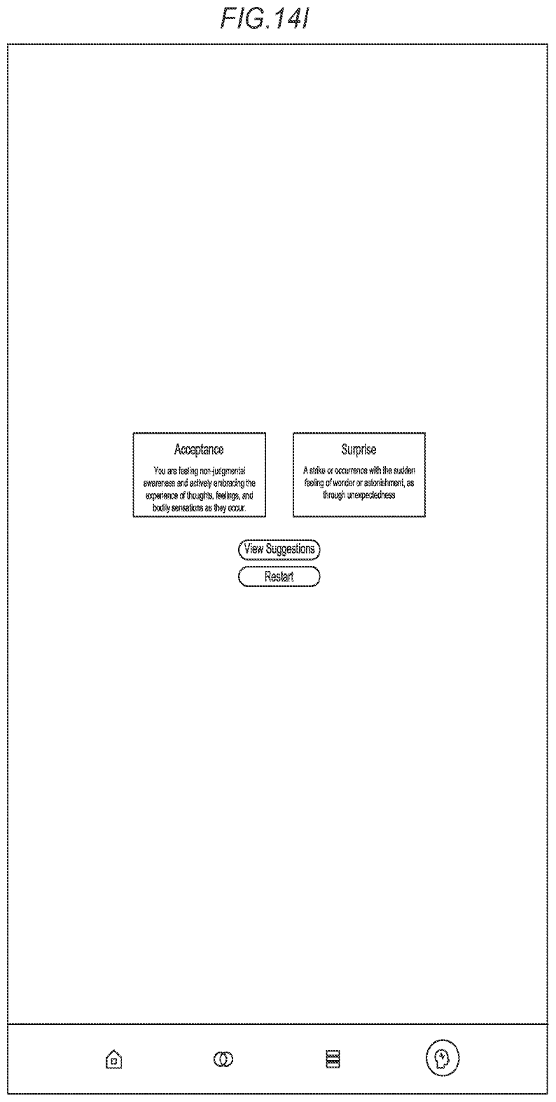

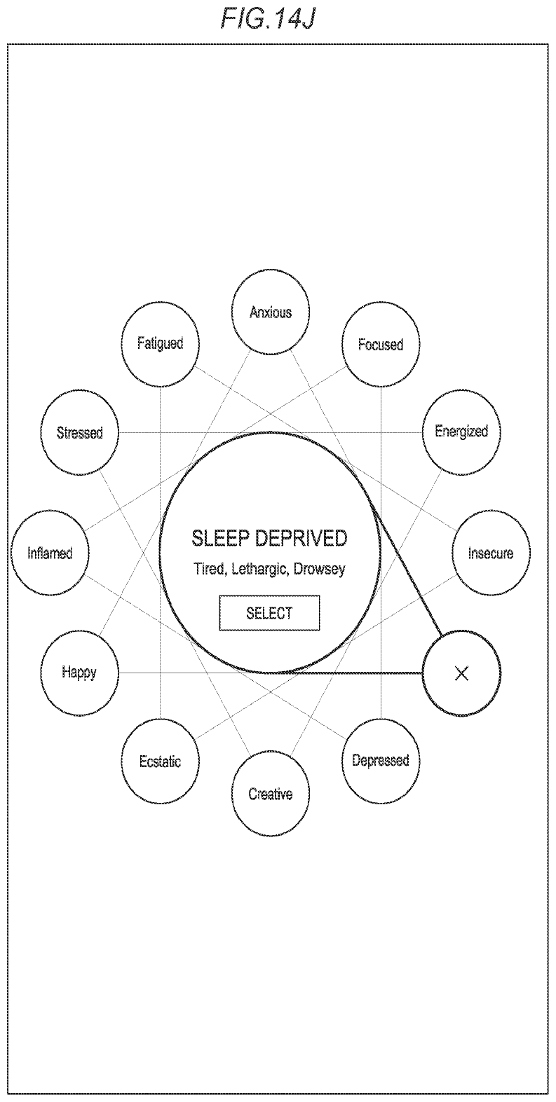

In some embodiments, the therapy bed 100 may be configured to play music and/or sounds that relate to a user's emotional state, the type of therapy the user desires, a target emotional state, and/or the like. The music may be selected using the emotional intelligence feature (e.g., machine learning component) and may be paired with different vibrational therapy routines. For example, as described further herein with reference to FIGS. 14A-14J, in some embodiments, users may be able to input one or more emotional state(s) (e.g., moods, emotions, mental states, etc.) into the system, or alternatively, the system can detect an emotional data (e.g., based on camera or sensor data). Based at least in part on this input, the emotional intelligence feature (e.g., machine learning model) may be configured to select music for the user that relate to the user's emotional state and pair vibrational therapy routines. In some embodiments, the emotional intelligence feature may select the music based on the brain wave frequency associated with the one or more emotional states (e.g., as empirically determined and mapped). Because human emotions are controlled by the brain, each emotion produces different brain wave frequencies. In some embodiments, the system may access a music library that includes music at a range of frequencies, where each music frequency is matched or paired with a corresponding brain wave frequency. When a user inputs an emotional state and/or the emotional state is otherwise determined/detected), the emotional intelligence feature may determine the corresponding brain wave frequency and select music to play that matches the frequency and vibrational routines that match the selected music. In some embodiments, the system may alter the frequency of both the music and/or the vibrations over the course of the therapy session to improve the emotional state of the user. For example, if a user indicates to the system that they are feeling sad, the emotional intelligence feature may initially select music that matches the brain wave frequency that corresponds to sadness. As the user begin s a therapy routine (e.g., a vibrational and sound therapy routine via the therapy bed 100), the emotional intelligence feature may change the music frequency over the course of the therapy session to change, adjust, or improve the user's emotional state. For example, the emotional intelligence feature may change the music and vibrations corresponding to a certain low frequency over time to increase the frequency in steps so that by the time the user completes the therapy, the music playing is at a higher frequency and the transducers 144 are producing high frequency vibrations that corresponds to a happy brain wave frequency. This feature allows the user to improve their emotional state through both music and completion of vibrational therapy. In some embodiments, the emotional intelligence feature may be configured to adjust a person's mood by a certain amount. For example, the emotional intelligence feature may be configured to adjust the mood of a user by a threshold. Depending on the original mood/emotional state of a user, it may be jarring for someone to move too quickly through different levels of therapy to improve their mood. For example, a user who is very low energy or sad may not want to undergo a therapy routine designed to adjust their emotional state to high energy and happy. Instead, a user may want a therapy routine designed to transition their emotional state to medium energy and contentment. In some embodiments, a user or an administrator may adjust the routine to target the desired threshold of a user. In some embodiments, the threshold can be determined based on personal data related to a user. In some embodiments, the threshold can be determined based on general data collected from a plurality of users. In some embodiments, the threshold may be determined by the machine learning component 216. In some embodiments, the threshold may relate to the total change between a current mood/emotional state of a user to a desired mood/emotional state of the user. In some embodiments, the threshold may relate to the total time spent in a therapy session. In some embodiments, the threshold may relate to a rate of change of the vibrational/music therapy over the course of a therapy routine. For example, the rate of change may be determined by the total change between a current and desired mood/emotional state of a user and the total time of the therapy routine. In some embodiments, a user may be asked to complete a second emotional check in following the completion of the therapy session. The emotional intelligence feature may use the second check in to improve the vibrational therapy selection and/or music section for future sessions.

FIGS. 14A-14J illustrate example interactive graphical user interfaces related to emotional intelligence-based sound and vibrational therapy, according to various embodiments of the present disclosure. In some embodiments, a user can be greeted with the interface via a UI associated with the therapy bed 100 or via a UI on a computing device (e.g., user device 202) and can select various elements of the interface to indicate their emotional state. As described herein, the system (e.g., ML model) can select sound and vibrational therapy suggestions to present to the user based in part on the indicated emotional state of the user and/or data about the user or the user's profile.

FIGS. 14A-14C illustrate interactive graphical user interfaces that show different emotional states a user can select. In some embodiments, the emotional states may be sorted into one or more categories including, for example, distress, energy, burnout, renewal, and/or the like. The emotional states may also be sorted into different grouping that may be displayed in, for example, a ring formation. For example, each of FIGS. 14A-14C display a different level of the emotional state ring. In some embodiments, a user may be able to progress through different levels of emotional states and select which of the presented emotional states apply to their emotional state.

FIGS. 14D-14I illustrates interactive graphical user interfaces showing a user selection of emotional states (e.g., acceptance). As shown, once the user makes the selection, the user may be able to confirm the selection by selecting the save button. FIG. 14E shows an embodiment of the next UI presented to the user having made one selection. In some embodiments, a user may be able to choose an option to combine emotions and make further selection of emotional states after a first section. As shown in FIGS. 14E-14G, a user may be able to progress through the rings to select one or more additional states.

FIG. 14H illustrates an interactive graphical user interfaces showing a user selection of an additional emotional states (e.g., surprise). Once the user makes the additional selection, the user may be given the option to save the additional selection and finish the emotional check-in.

FIG. 14I illustrates an interactive graphical user interface showing the one or more emotional states selected by the user. As shown, in some embodiments, the UI may include some further information about the selected emotional states. In some embodiments, users may be given the option to view suggestions related to the emotional check-in, such as, for example, music options, vibration options, light display option, visualization options, and/or the like. Users may also be given the option to restart the check-in by selecting new emotional states. As described herein, each emotional state check-in can be logged by the system and users'trends and historical selections that may be accessible by the user. FIG. 14 J illustrates a selection by a user.

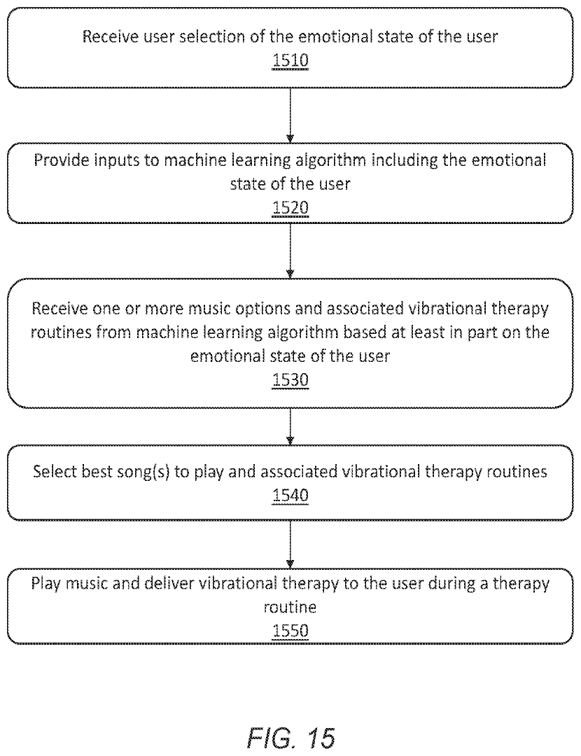

FIG. 15 illustrates a flow diagram of an example method of determining music for a therapy routine based on an emotional state of a user. Emotional state, as the term is used herein, is intended to be a broad term and may include a user's mood, emotions, mental state, and/or the like at or during a given point in time (e.g., at a specific moment, over a day, over a week, or the like). Embodiments and aspects of the example method are discussed herein, for example, with reference to at least FIGS. 14A-14J. It is recognized that there are other embodiments of the method of FIG. 15 which may exclude some of the blocks shown and/or include additional blocks not shown. Additionally, the blocks discussed may be combined, separated into sub-blocks, and/or rearranged to be completed in a different order and/or in parallel.

At block 1510, the system receives a user selection of the emotional state of the user and/or determines the user's emotional state by using data collected by one or more cameras or sensors (e.g., as well as using a machine learning model to make determinations based on the collected data). In some embodiments, a user may make a selection via a UI on the machine, while in other embodiments, a user may make a selection via another computing device in wired or wireless communication with the system. For example, a user may input a selection using an app associated with the therapy bed 100 via the user device 202. In some embodiments, the user may be able to select one or more emotional states (e.g., surprise, acceptance, anxious, happy, sad, depressed, and/or the like) and the system may combine the emotional states for further analyses. As shown in FIGS. 14A-14J, in some embodiments, the emotional states may be sorted into one or more categories including, for example, distress, energy, burnout, renewal, and/or the like. The emotional states may also be sorted into different grouping that may be displayed in, for example a ring formation. In some embodiments, a user may be able to progress through different levels of emotional states and select which of the presented emotional states apply to their emotional state. In some embodiments, users may be able to input additional emotional states into the system by, for example, selecting a portion of the UI (e.g., “not listed?”) and input their emotional state (e.g., by typing or saying their emotional state out loud). After a user selects one emotional state, the user may be given the option to save the emotional state. In some embodiments, once one emotional state is input, the user may be given the option to select an additional emotional state and combine the emotions. In some embodiments, a user may indicate to the system that they have completed their selections by selecting, for example a “finish” button. In some embodiments, as a user selects an emotional state, information about the emotional state may be displayed for the user.

At block 1520, the system provides inputs to a machine learning algorithm/model (e.g., an emotional intelligence model), including the emotional state of the user. In some embodiments, the inputs may include one or more of: data input into the system by the user, such as, for example, health goals and intentions, medical information, user specific information (e.g., age, sex, weight, and/or the like), user emotional state, historical user emotional state, user pain selections, and/or the like, as well as data generated by the system or third party systems, such as movement assessment data, thermal assessment data, user emotional state, other data collected by the system (e.g., historical routine information), and/or the like. In some embodiments, a user may have indicated to the system (e.g., during account/profile creation) one or more music style preferences, favorite bands, and/or the like that may also be input into the machine learning model. In some embodiments, the machine learning uses the input emotional state(s) of the user and identifies one or more brain wave frequencies associated with the one or more emotional states. Because human emotions are controlled by the brain, each emotion produces different brain wave frequencies. In some embodiments, the machine learning model may correlate each input emotional state to the corresponding brain wave frequency. Based on the brain wave frequencies, the machine learning model may access a music library that includes music at a range of frequencies, where each music frequency matches a corresponding brain wave frequency. Additionally, in some embodiments, each music in the music library is paired with a vibrational therapy routine that can deliver different patterns, frequencies, intensities and/or the like via the transducers 144 of the therapy bed 100. The machine learning model may then generate a list of one or more songs and vibrational therapy routines that have frequencies that match the emotional state (e.g., brain waves) of the user. In some embodiments, the machine learning model may further refine the list of one or more songs based on the other data input into the model. For example, the machine learning model may reorganize or rank the songs based on the other data including the user's music preferences, historical music selections, and/or the like.

In some embodiments, the machine learning model may also generate one or more songs to create a path of songs and vibrations to a target final song and final vibration. For example, the machine learning model may identify a target emotional state for the user (e.g., happy) and may select a final song and vibration that has a corresponding frequency to the brain waves associated with the target emotional state. Based on the starting and final songs and vibrations, the machine learning model may create a path of songs and vibrations that include music and vibrations that transition from the starting frequency to the target frequency. For example, where the user's emotional state corresponds to low frequencies, and the target emotional state corresponds to high frequencies, the path of songs would include one or more songs that progressively increase in frequency (e.g., first song is low frequency, next song is a higher frequency, next song is high frequency than the previous song, etc.). Similarly, the vibrations delivered by the transducers 144 may increase in frequency and/or intensity as the user progress through the therapy. In some embodiments, the number of songs selected and/or the length of the playlist selected corresponds to the length of time for the user to complete the intended therapy routine scheduled for that day. For example, if the user indicated to the system that they were going to complete a 45-minute therapy routine or were scheduled for a 45-minute therapy routine, the machine learning model generated playlist may include enough music to last for the entire routine (i.e., approximately 45 minutes). In some embodiments, the machine learning model may generate more than one playlist that includes music and vibrations to complete the entire path.

At block 1530, the system can receive one or more music options and associated vibrational therapy routines from the machine learning algorithm based at least in part on the emotional state of the user. For example, as described above, the machine learning model may output one or more songs and vibration patterns and/or one or more playlists that include a path of songs and vibrations from an emotional state to a target emotional state.

At block 1540, the system selects the best song(s) and associated vibrational therapy routines. In some embodiments, the system may select a top ranked machine learning model playlist to play for the user. In other embodiments, the one or more songs and/or playlists may be presented to the user (e.g., via a UI displayed on user device 202) and the user may select which songs/playlist corresponding to a desired therapy for the therapy bed 100 to generate.

At block 1550, the system can play music for the user and produce vibrations during the routine (e.g., by headphones, by speakers 162 and/or speakers 164, and/or by transducers 144). In some embodiments, a user may be asked to complete a second emotional check-in following the completion of the routine. The machine learning model may use the second check in to improve music section for future sessions for the user and/or other users (e.g., by improving the machine learning model, where updates can be shared among devices belonging to different users).

In some embodiments, the method of FIG. 15 may be automatically generated and presented to the user as part of a daily emotional check-in (e.g., after the user logs in or begins interacting with the system). In other embodiments, the method of FIG. 15 may be automatically generated and presented to the user after the user indicated they wish to participate in a vibrational/sound therapy session. In some embodiments, a user may be able to complete one or more emotional check-ins at any point during their interaction with the system. In some embodiments, emotional check-ins can occur via other devices (e.g., user device 202 by an app). The system may store the user emotional states so that the user or system can track the emotional states of the user over time.

### VII. Computer Systems

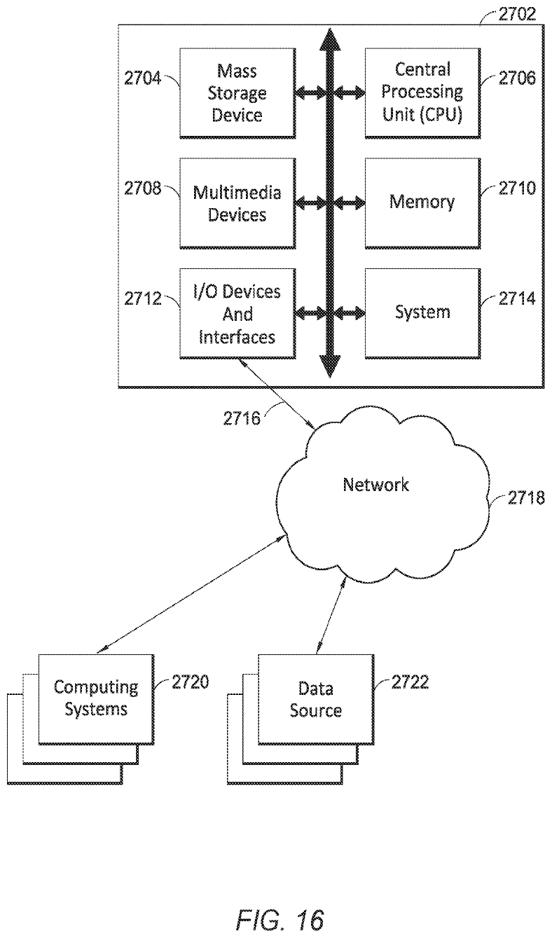

FIG. 16 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 16. The example computer system 2702 is in communication with one or more computing systems 2720 and/or one or more data sources 2722 via one or more networks 2718. While FIG. 16 illustrates an embodiment of a computing system 2702, it is recognized that the functionality provided for in the components and modules of computer system 2702 may be combined into fewer components and modules, or further separated into additional components and modules.

The computer system 2702 can comprise a programming module 2714 that carries out the functions, methods, acts, and/or processes described herein. The programming module 2714 is executed on the computer system 2702 by a central processing unit 2706 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 2702 includes one or more processing units (CPU) 2706, which may comprise a microprocessor. The computer system 2702 further includes a physical memory 2710, such as random-access memory (RAM) for temporary storage of information, a read only memory (ROM) for permanent storage of information, and a mass storage device 2704, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 2702 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 2702 includes one or more input/output (I/O) devices and interfaces 2712, such as a keyboard, mouse, touch pad, and printer. The I/O devices and interfaces 2712 can include one or more display devices, such as a monitor, which allows the visual presentation of data to a user. More particularly, a display device provides for the presentation of GUIs as application software data, and multi-media presentations, for example. The I/O devices and interfaces 2712 can also provide a communications interface to various external devices. The computer system 2702 may comprise one or more multi-media devices 2708, such as speakers, video cards, graphics accelerators, and microphones, for example.

The computer system 2702 may run on a variety of computing devices, such as a server, a Windows server, a Structure Query Language server, a Unix Server, a personal computer, a laptop computer, a smart phone, a personal digital assistant, a tablet, and so forth. Servers may include a variety of servers such as database servers (for example, Oracle, DB2, Informix, Microsoft SQL Server, MySQL, or Ingres), application servers, data loader servers, or web servers. In addition, the servers may run a variety of software for data visualization, distributed file systems, distributed processing, web portals, enterprise workflow, form management, and so forth. In other embodiments, the computer system 2702 may run on a cluster computer system, a mainframe computer system and/or other computing system suitable for controlling and/or communicating with large databases, performing high volume transaction processing, and generating reports from large databases. The computing system 2702 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 2702 illustrated in FIG. 16 is coupled to a network 2718, such as a LAN, WAN, or the Internet via a communication link 2716 (wired, wireless, or a combination thereof). Network 2718 communicates with various computing devices and/or other electronic devices. Network 2718 is communicating with one or more computing systems 2720 and one or more data sources 2722. The programming module 2714 may access or may be accessed by computing systems 2720 and/or data sources 2722 through a web-enabled user access point. Connections may be a direct physical connection, a virtual connection, and other connection type. The web-enabled user access point may comprise a browser module that uses text, graphics, audio, video, and other media to present data and to allow interaction with data via the network 2718.

Access to the programming module 2714 of the computer system 2702 by computing systems 2720 and/or by data sources 2722 may be through a web-enabled user access point such as the computing systems'2720 or data source's 2722 personal computer, cellular phone, smartphone, laptop, tablet computer, e-reader device, audio player, or another device capable of connecting to the network 2718. Such a device may have a browser module that is implemented as a module that uses text, graphics, audio, video, and other media to present data and to allow interaction with data via the network 2718.

The output module may be implemented as a combination of an all-points addressable display such as a cathode ray tube (CRT), a liquid crystal display (LCD), a plasma display, or other types and/or combinations of displays. The output module may be implemented to communicate with input devices 2712 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 2702 may comprise a physical or logical connection established between a remote microprocessor and a mainframe host computer for the express purpose of uploading, downloading, or viewing interactive data and databases on-line in real time. The remote microprocessor may be operated by an entity operating the computer system 2702, including the client server systems or the main server system, an/or may be operated by one or more of the data sources 2722 and/or one or more of the computing systems 2720. In some embodiments, terminal emulation software may be used on the microprocessor for participating in the micro-mainframe link.

In some embodiments, computing systems 2720 who are internal to an entity operating the computer system 2702 may access the programming module 2714 internally as an application or process run by the CPU 2706.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL ca specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, or the like. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 2702 may include one or more internal and/or external data sources (for example, data sources 2722). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MogoDB Atlas, Amazon Aurora, Amazon DynamoDB, Amazon Redshift, Amazon ElastiCache, Amazon MemoryDB for Redis, Amazon DocumentDB, Amazon Keyspaces, Amazon EKS, Amazon Neptune, Amazon Timestream, or Amazon QLDB), a non-relational database, or a record-based database.

The computer system 2702 may also access one or more databases 2722. The databases 2722 may be stored in a database or data repository. The computer system 2702 may access the one or more databases 2722 through a network 2718 or may directly access the database or data repository through I/O devices and interfaces 2712. The data repository storing the one or more databases 2722 may reside within the computer system 2702.

### VIII. Additional Implementation Details and Embodiments

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums).

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Java, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

As described above, in various embodiments certain functionality may be accessible by a user through a web-based viewer (such as a web browser), or other suitable software program. In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain embodiments, one or more of the computing devices and/or systems of the disclosure may include mobile computing devices, and user interfaces may be accessible through such mobile computing devices (for example, smartphones and/or tablets).

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain embodiments require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general purpose computer comprising one or more processors should not be interpreted as excluding other computer components, and may possibly include such components as memory, input/output devices, and/or network interfaces, among others.

While the above detailed description has shown, described, and pointed out novel features as applied to various embodiments, it may be understood that various omissions, substitutions, and changes in the form and details of the devices or processes illustrated may be made without departing from the spirit of the disclosure. As may be recognized, certain embodiments of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

### IX. Example Clauses

Various examples of systems and methods relating to a therapy bed are found in the following clauses:

Clause 1. A computer-implement method comprising, by one or more hardware processors executing program instructions: receiving, selection of a first therapy routine; generating, instructions configured to play music; and generating, instructions configured to produce vibrations.

Clause 2. The computer-implement method of Clause 1, further comprising: generating, instructions configured to produce light associated with the music.

Clause 3. The computer-implement method of Clause 1 or Clause 2, wherein selection of the first therapy routine includes receiving a selection of a user emotional state.

Clause 4. The computer-implement method of any of Clauses 1 to 3, wherein a vibrational frequency of the vibrations is related to a music frequency of the music.

Clause 5. The computer-implement method of Clause 4, wherein the music frequency changes over time.

Clause 6. The computer-implement method of any of Clause 4 or Clause 5, wherein the vibrational frequency changes over time.

Clause 7. A system comprising: a bed portion configured to receive and support a user; a plurality of transducers positioned below the bed portion, wherein the plurality of transducers are configured to produce vibrations; and a plurality of speakers.

Clause 8. The system of Clause 7, further comprising a light system, wherein the light system extends at least a portion of the way around the bed portion.

Clause 9. The system of Clause 7 or Clause 8, further comprising a first control system and a second control system.

Clause 10. The system of Clause 9, wherein the first control system controls the plurality of speakers and/or the plurality of transducers.

Clause 11. The system of any of Clauses 7 to 10, wherein the bed portion comprises a resiliently compressible material.

Clause 12. The system of any of Clauses 7 to 11, further comprising a frame configured to support the bed portion.

Clause 13. The system of Clause 12, wherein the frame comprises a first wing and a second wing, wherein the first wing and the second wing are configured to adjust a shape of the bed portion.

Clause 14. The system of Clause 13, wherein the first wing and the second wing are configured to move from a first position to a second position, wherein in the first position, the bed portion is approximately flat, wherein in the second position, the bed portion comprises a recess.

Clause 15. The system of any of Clauses 7 to 14, wherein the plurality of speakers are positioned on one or more brackets, wherein the one or more brackets direct the plurality of speakers towards the user's head.

Clause 16. The system of Clause 12, further comprising a light system, wherein the light system extends at least a portion of the way around the frame.

Clause 17. The system of any of Clauses 7 to 16, wherein the plurality of speakers are positioned on one or more brackets.

Clause 18. The system of any of Clauses 7 to 17, wherein the plurality of speakers comprises one or more top speakers and one or more side speakers.

Clause 19. The system of Clause 18, wherein the one or more side speakers comprise a first side speaker positioned on a first bracket and a second side speaker positioned on a second bracket, wherein the first bracket directs audio from the first side speaker towards the user's first ear and the second bracket directs audio from the second side speaker towards the user's second ear.

Clause 20. The system of Clause 18 or Clause 19, wherein the one or more top speakers are positioned on a third bracket, wherein the third bracket directs audio from the one or more top speakers towards a top of the user's head.

What is claimed is:

1. A system comprising:
a bed portion configured to receive and support a user;
a plurality of transducers positioned below the bed portion, wherein the plurality of transducers are configured to produce vibrations; and
a plurality of speakers.

2. The system of claim 1, further comprising a light system, wherein the light system extends at least a portion of the way around the bed portion.

3. The system of claim 1, further comprising a first control system and a second control system.

4. The system of claim 3, wherein the first control system controls the plurality of speakers and/or the plurality of transducers.

5. The system of claim 1, wherein the bed portion comprises a resiliently compressible material.

6. The system of claim 1, further comprising a frame configured to support the bed portion.

7. The system of claim 6, wherein the frame comprises a first wing and a second wing, wherein the first wing and the second wing are configured to adjust a shape of the bed portion.

8. The system of claim 7, wherein the first wing and the second wing are configured to move from a first position to a second position, wherein in the first position, the bed portion is approximately flat, wherein in the second position, the bed portion comprises a recess.

9. The system of claim 1, wherein the plurality of speakers are positioned on one or more brackets, wherein the one or more brackets direct the plurality of speakers towards the user's head.

10. The system of claim 6, further comprising a light system, wherein the light system extends at least a portion of the way around the frame.

11. The system of claim 1, wherein the plurality of speakers are positioned on one or more brackets.

12. The system of claim 1, wherein the plurality of speakers comprises one or more top speakers and one or more side speakers.

13. The system of claim 12, wherein the one or more side speakers comprise a first side speaker positioned on a first bracket and a second side speaker positioned on a second bracket, wherein the first bracket directs audio from the first side speaker towards the user's first ear and the second bracket directs audio from the second side speaker towards the user's second ear.

14. The system of claim 12, wherein the one or more top speakers are positioned on a third bracket, wherein the third bracket directs audio from the one or more top speakers towards a top of the user's head.

15. A computer-implement method comprising, by one or more hardware processors executing program instructions:
receiving, selection of a first therapy routine;
generating, instructions configured to play music; and
generating, instructions configured to produce vibrations.

16. The computer-implement method of claim 15, further comprising:
generating, instructions configured to produce light associated with the music.

17. The computer-implement method of claim 15, wherein selection of the first therapy routine includes receiving a selection of a user emotional state.

18. The computer-implement method of claim 15, wherein a vibrational frequency of the vibrations is related to a music frequency of the music.

19. The computer-implement method of claim 18, wherein the music frequency changes over time.

20. The computer-implement method of claim 18, wherein the vibrational frequency changes over time.
